(19) United States
(12) Patent Application Publication
(10) Pub. No.: US 2025/0005744 A1
(43) Pub. Date: Jan. 2, 2025

(54) A CORONARY ARTERY DISEASE ANALYSIS SYSTEM

(71) Applicant: ARTRYA LIMITED, Perth (AU)

(72) Inventors: Abdul IHDAYHID, Ardross (AU); Casey CLIFTON, Subiaco (AU); Girish DWIVEDI, Dalkeith (AU); Jack JOYNER, Floreat (AU); John KONSTANTOPOULOS, Burns Beach (AU); Julien FLACK, Swanbourne (AU)

(73) Assignee: ARTRYA LIMITED, Perth, WA (AU)

(21) Appl. No.: 18/292,843

(22) PCT Filed: Jul. 12, 2022
(86) PCT No.: PCT/AU2022/050727

§ 371 Date: Jan. 26, 2024

(30) Foreign Application Priority Data
Jul. 28, 2021 (AU) 2021902323
Aug. 25, 2021 (AU) 2021221669

(51) Int. Cl. G06T 7/00; A61B 6/50; G16H 30/40; G16H 40/63

(52) U.S. Cl. CPC G06T 7/0012; G16H 30/40; G16H 40/63; A61B 6/503; A61B 6/504; G06T 2200/24; G06T 2207/10081; G06T 2207/30048; G06T 2207/30096; G06T 2207/30101

(57) ABSTRACT

A coronary artery disease (CAD) analysis system is disclosed that comprises a CAD analysis device and a user interface. The CAD analysis device is arranged to analyse received patient CT scan data and produce CAD analysis data indicative of presence and characterisation of coronary artery disease in the patient CT scan data. The CAD analysis data is indicative of at least one individual stenosis lesion on a coronary artery and a characterisation of the stenosis lesion, and the CAD analysis system is arranged to identify a start location and an end location of the individual stenosis lesion on the coronary artery. The user interface displays a model of coronary arteries of a patient based on the patient CT scan data, and the model is configured to visually indicate an individual stenosis lesion and the individual stenosis lesion characterisation to a user so that the user is able to identify presence and characterisation of each individual stenosis lesion based on the visual indication.

### CROSS REFERENCE TO RELATED APPLICATIONS

This application is a national stage filing under section 371 of International Application No. PCT/AU2022/050727, filed on Jul. 12, 2022, and published on Feb. 2, 2023, as WO 2023/004451, which claims priority to Australian Patent Application No. 2021902323 filed on Jul. 28, 2021, and to Australian Patent Application No. 2021221669 filed on Aug. 25, 2021. The entire contents of each application are incorporated herein by reference in their entireties.

### FIELD OF THE INVENTION

The present disclosure relates to a coronary artery disease analysis system.

### BACKGROUND OF THE INVENTION

Coronary Artery Calcium (CAC) scores are an important indicator of Coronary Artery Disease (CAD) and are commonly calculated using Agatston's method of density weighted area calculation.

Atherosclerosis is a disease of the coronary arteries wherein atheromatous plaque (“plaque”) accumulates abnormally in the inner layer of an arterial wall. Significant accumulation of plaque can cause a narrowing of an artery, referred to as arterial stenosis, and consequently a reduction of blood flow. Significant arterial stenosis in the context of coronary arteries can result in heart attack and death. Accumulation of vulnerable plaque in a coronary artery also poses a significant health risk as it tends to be unstable and prone to rupture, which can cause an acute cardiovascular event such as a heart attack or a stroke.

It is known to provide a coronary artery disease (CAD) analysis system that includes a user interface usable to communicate patient coronary artery disease related information to a user.

However, such CAD analysis system can be relatively cumbersome to use and in some cases it is difficult for a user to quickly obtain relevant CAD information for a patient using the system.

### SUMMARY OF THE INVENTION

In accordance with a first aspect of the present invention, there is provided a coronary artery disease (CAD) analysis system comprising:
a CAD analysis device arranged to analyse received patient CT scan data and produce CAD analysis data indicative of presence and characterisation of coronary artery disease in the patient CT scan data, the CAD analysis data indicative of at least one individual stenosis lesion on a coronary artery and a characterisation of the stenosis lesion;

the CAD analysis system arranged to identify a start location and an end location of the individual stenosis lesion on the coronary artery by identifying start and end slices of the individual stenosis lesion, and the CAD analysis system arranged to categorise all slices between the start and end slices as belonging to the individual stenosis lesion; and
the CAD analysis system comprising a user interface that displays a model of coronary arteries of a patient based on the patient CT scan data, the model configured to visually indicate an individual stenosis lesion and the individual stenosis lesion characterisation to a user so that the user is able to identify presence and characterisation of each individual stenosis lesion based on the visual indication;
wherein the characterisation of the individual stenosis lesion includes a stenosis level for the individual stenosis lesion.

In an embodiment, the characterisation also includes:
an indication of vulnerable plaque presence and vulnerable plaque type;
an indication of plaque presence and plaque type;
a lesion number; and/or
an artery slice number.

In an embodiment, the stenosis level is visually communicated to the user using colour.

In an embodiment, the stenosis level is visually communicated by displaying a portion of a coronary artery associated with the individual stenosis lesion in a defined colour of a plurality of colours of a stenosis level colour key, the defined colour corresponding to the stenosis level of the lesion.

In an embodiment, the visual indication of the individual stenosis lesion and the individual stenosis lesion characterisation are displayed in response to user input.

In an embodiment, the visual indication of the individual stenosis lesion and the individual stenosis lesion characterisation are displayed in response to user selection of a location on a coronary artery considered to correspond to a stenosis lesion.

In an embodiment, the CAD analysis device is arranged to analyse received patient CT scan data associated with at least a location on a coronary artery selected by a user and produce CAD analysis data indicative of presence and characterisation of coronary artery disease at least at the selected location on the coronary artery in response to the selection of the location by the user.

In an embodiment, the model of coronary arteries includes a first vessel slice identifier arranged to indicate a selected slice of a coronary artery. The first vessel slice identifier may include a graphical identifier representing a frame around the coronary artery at a location on the coronary artery corresponding to the selected slice.

In an embodiment, the user interface is arranged to indicate a most significant lesion.

In an embodiment, the displayed model of coronary arteries is a 3D model of coronary arteries, the orientation of the 3D model modifiable by a user about 1, 2 or 3 mutually orthogonal axes.

In an embodiment, the user interface includes a multiplanar reconstruction (MPR) representation of at least a selected coronary artery. The MPR representation may include a second vessel slice identifier arranged to indicate a selected slice of the selected coronary artery.

In an embodiment, the second vessel slice identifier includes a graphical identifier representing a line through the selected coronary artery at a location on the selected coronary artery corresponding to the selected slice.

In an embodiment, the first and second vessel slice identifiers are synchronised such that selection of a vessel slice using one of the first and second vessel slice identifiers causes a corresponding vessel slice to be selected using the other of the first and second vessel slice identifiers.

In an embodiment, the MPR representation is a curved multiplanar reconstruction (CPR) or a straightened multiplanar reconstruction (SPR).

In an embodiment, the user interface further includes an axial slice representation of a selected coronary artery at a selected coronary artery slice. The axial slice representation may include inner and outer vessel wall annotations.

In an embodiment, for the axial slice representation, the user interface further includes stenosis lesion specific information for the stenosis lesion with which the selected slice is associated.

In an embodiment, the lesion specific information associated with the selected slice includes:
a stenosis level of the stenosis lesion with which the selected slice is associated;
plaque type information for plaque present on the stenosis lesion with which the selected slice is associated; and/or
vulnerable plaque type information for vulnerable plaque present on the stenosis lesion with which the selected slice is associated.

In an embodiment, the stenosis level of the stenosis lesion with which the selected slice is associated is communicated by displaying a portion of the MPR representation corresponding to the stenosis lesion using the colour used to display the stenosis lesion on the model of the coronary arteries.

In an embodiment, the stenosis level of the stenosis lesion with which the selected slice is associated is communicated by displaying text indicative of the stenosis level using the colour used to display the stenosis lesion on the model of the coronary arteries.

In an embodiment, the MPR representation includes a proximal vessel slice identifier arranged to indicate a proximal slice of the selected coronary artery, the proximal slice located proximal to the aorta than the selected vessel slice identifier, and the user interface further includes a proximal slice representation of the selected coronary artery at the proximal coronary artery slice.

In an embodiment, the MPR representation includes a distal vessel slice identifier arranged to indicate a distal slice of the selected coronary artery, the distal slice located distal to the aorta than the selected vessel slice identifier, and the user interface further includes a distal slice representation of the selected coronary artery at the distal coronary artery slice.

In an embodiment, the user interface is arranged to display a coronary artery centreline in response to user input. The path of the centreline may be editable by a user.

In an embodiment, the user interface is arranged to enable a user to add a new centreline associated with a coronary artery.

In an embodiment, the user interface is arranged to display representations of calcified volumes on the model of coronary arteries in response to user input.

In an embodiment, the user interface is arranged to display information indicative of locations of vulnerable plaque on the model of coronary arteries in response to user input.

In an embodiment, the user interface is arranged to display a snapshot of displayed information and to facilitate addition of user annotations to the snapshot.

In an embodiment, the user interface is arranged to display summary patient analysis information.

In an embodiment, the summary patient analysis information includes:
a maximum stenosis level indicative of the maximum stenosis level of all lesions associated with the CT scan data;
an indication of all vulnerable plaques present in the CT scan data;
an indication of plaque presence and plaque type;
a CAC score;
a CAD-RADS classification; and/or
a lesion involvement score indicative of the number of lesions in the CT scan data.

In an embodiment, at least some CAD relevant information displayed on the user interface is editable by a user.

In an embodiment, the user interface is arranged to simultaneously display multiple CT volume representations of a CT volume associated with the patient CT scan data, each CT volume representation taken along a plane extending through the CT volume at a different orientation, and the multiple displayed CT volume representations having a common CT volume location.

In an embodiment, the CT volume representations correspond to planes extending through the CT volume at mutually orthogonal orientations.

In an embodiment, the CT volume representations correspond to an axial plane, a coronal plane and a sagittal plane.

In an embodiment, the user interface is arranged to display plane indicia on a CT volume representation, the plane indicia indicative of a plane associated with another CT volume representation, and to enable a user to interact with the plane indicia to modify the plane associated with the other CT volume representation and thereby the displayed other CT volume representation.

In an embodiment, the plane indicia is modifiable so as to change the orientation of a plane associated with the plane indicia.

In an embodiment, the plane indicia is a line normal to the plane associated with the other CT volume representation.

In an embodiment, the user interface is arranged to enable a user to modify at least one CT volume representation of the multiple CT volume representations of the CT volume so as to selectively display a CT volume representation associated with a different plane parallel to a plane associated with a current CT volume representation.

In an embodiment, the user interface is arranged to modify the location of plane indicia on another CT volume representation in response to display of a different CT volume representation associated with a different plane parallel to the plane of current CT volume representation.

In an embodiment, the user interface is arranged to display a vessel MPR representation of a selected vessel with the multiple CT volume representations of the CT volume, wherein the common CT volume location is a selected location on the vessel MPR.

In an embodiment, the user interface is arranged to enable a user to move the selected location on the vessel MPR, and to change the multiple displayed CT volume representations of the CT volume in synchronisation with the selected location on the vessel MPR so that the common CT location changes in accordance with the moved location on the vessel MPR.

In an embodiment, the CAD analysis system is arranged to enable a user to modify and/or add to displayed CAD analysis information, and in response the analysis device is arranged to reanalyse the patient CT scan data in consideration of the modification and/or addition.

In an embodiment, the user interface includes a plurality of viewing panes, each viewing pane associated with particular information and/or a particular representation of the information, and the CAD analysis system arranged such that the displayed viewing panes are customisable.

In an embodiment, the displayed viewing panes are customisable by the user.

In an embodiment, the displayed viewing panes are customised in response to selected functionality.

In an embodiment, the user interface is arranged to display plaque on a coronary artery and a visual indication of plaque type by assigning a different colour to each of a plurality of plaque types and displaying the plaque in a colour that corresponds to the determined plaque type.

In accordance with a second aspect of the present invention, there is provided a coronary artery disease (CAD) analysis system, the CAD analysis system comprising:
a CAD analysis device arranged to receive CAD data indicative of presence of calcified plaque on coronary arteries in patient CT scan data; and
a user interface arranged to:
   display a scroll bar having a user controllable position indicator and coronary artery calcium indicia disposed adjacent the scroll bar, the location of the position indicator relative to the scroll bar indicating a respective position along an axis of a CT volume associated with the patient CT scan data, and each coronary artery calcium indicium indicative of a total amount of coronary artery calcification at an axial location of the CT scan data corresponding to the relative position of the position indicator on the scroll bar; and
   display information indicative of coronary artery calcification at an axial location of the CT volume and associated with a coronary artery calcium indicium when the position indicator is disposed adjacent the coronary artery calcium indicium.

In an embodiment, each calcium indicium includes a graphical indicator, wherein a dimension of the graphical indicator is indicative of a size of the associated calcified volume.

In an embodiment, the graphical indicator is a line and the length of the line is indicative of the size of the associated calcified volume.

In an embodiment, a colour of the calcium indicium is indicative of a coronary artery on which the calcified volume is located.

In an embodiment, the colour used for the calcium indicium is also used for the calcified volume associated with the calcium indicium.

In an embodiment, a vessel label is displayed adjacent a displayed calcified volume.

In an embodiment, the vessel label is displayed adjacent a displayed calcified volume in response to user input.

In an embodiment, the coronary artery label associated with a displayed calcified volume is editable by a user to change the associated coronary artery to a different coronary artery.

In an embodiment, non-coronary artery calcium is displayed in a different colour to the calcified volumes displayed on the coronary arteries.

### BRIEF DESCRIPTION OF THE DRAWINGS

The present invention will now be described by way of example only with reference to the accompanying drawings, in which:

FIG. 1 is a schematic block diagram of a coronary artery disease (CAD) analysis system in accordance with an embodiment of the present invention;

FIG. 2 is a schematic block diagram of a coronary artery disease analysis device of the system shown in FIG. 1;

FIG. 3 is a diagrammatic representation of a scan menu screen presented to a user after the user has logged into the CAD analysis system;

FIG. 4 is a diagrammatic representation of a patient overview screen of a CAD analysis system according to an embodiment of the present invention;

FIG. 5 is a representation of a patient analysis overview pane of the patient overview screen shown in FIG. 4;

FIG. 6 is a representation of a 3D model pane of the patient overview screen shown in FIG. 4;

FIG. 7 is a representation of a multiplanar reconstruction (MPR) pane of the patient overview screen shown in FIG. 4;

FIG. 8 is a representation of a vessel slice pane of the patient overview screen shown in FIG. 4;

FIG. 9 is an enlarged view of a portion of a 3D model shown in FIG. 6 and showing a selected artery slice;

FIG. 10 is an enlarged view of a portion of the 3D model shown in FIG. 6 showing a different selected artery slice;

FIG. 11 is an axial representation of the selected artery slice shown in FIG. 10;

FIG. 12 is an enlarged view of a portion of the 3D model shown in FIG. 6 showing a different selected artery slice;

FIG. 13 is an axial representation of the selected artery slice shown in FIG. 12;

FIG. 14 is an enlarged view of a portion of the 3D model shown in FIG. 6 showing a different selected artery slice;

FIG. 15 is an axial representation of the selected artery slice shown in FIG. 14;

FIG. 16 is a representation of a stenosis level selection list usable to edit the stenosis level of a stenosis lesion associated with a displayed selected artery slice;

FIG. 17 is a representation of a calcification selection list usable to edit the calcification associated with a displayed selected artery slice;

FIG. 18 is a representation of the MPR pane shown in FIG. 7 and including an artery centreline;

FIG. 19 is a representation of an artery shown in the MPR pane with the artery transformed so as to appear linear;

FIG. 20 is a representation of the 3D model pane shown in FIG. 6 showing calcified volumes on a 3D model in the 3D model pane;

FIG. 21 is a representation of the 3D model pane shown in FIG. 6 showing vulnerable plaque locations on a 3D model in the 3D model pane;

FIG. 22 is a diagrammatic representation of a CT volume screen of the CAD analysis tool showing CT volume non-contrast results;

FIG. 23 is a diagrammatic representation of a multi-view screen of the CAD analysis tool showing contrast results;

FIG. 24 is a representation of an example CT volume screen shown in FIG. 22;

FIG. 25 is a representation of a scroll bar pane of the CT volume screen shown in FIG. 22;

FIG. 26 is a representation of a portion of the CT volume in FIG. 24 showing a selected scroll bar position and associated CT volume slice and calcified volume;

FIG. 27 is an enlarged representation of a calcified volume including a coronary artery label;

FIG. 28 is an enlarged representation of a calcified volume including a coronary artery selection list;

FIG. 29 is a representation of a further CT volume slice associated with a different selected scroll bar position;

FIG. 30 is a representation of a screenshot annotation screen of the CAD analysis tool;

FIG. 31 is an example representation of a multi-view screen with axial, coronal and sagittal normal lines shown in a first position and a vessel slice indicator shown in a first position;

FIG. 32 is an example representation of a multi-view screen with axial, coronal and sagittal normal lines shown in a first position and a vessel slice indicator shown in a second position;

FIG. 33 is an example representation of a multi-view screen with axial, coronal and sagittal normal lines shown in a second position and a vessel slice indicator shown in a first position;

FIG. 34 is a diagrammatic representation of a report screen of the CAD analysis system;

FIG. 35 is a representation of a patient information pane of the report screen shown in FIG. 34;

FIG. 36 is a representation of a patient analysis overview section of the report screen shown in FIG. 34;

FIG. 37 is a representation of a report commentary pane of the report screen shown in FIG. 34;

FIG. 38 is a representation of a status and edit pane of the report screen shown in FIG. 34;

FIG. 39 is a diagrammatic representation of a patient overview screen of a CAD analysis system according to an alternative embodiment of the present invention;

FIG. 40 is a representation of a patient analysis overview pane of the patient overview screen shown in FIG. 39 prior to interaction with a user;

FIG. 41 is a representation of a vessel slice pane of the patient overview screen shown in FIG. 39 prior to interaction with a user;

FIG. 42 is a representation of a multiplanar reconstruction (MPR) pane of the patient overview screen shown in FIG. 39;

FIG. 43 is a representation of the vessel slice pane shown in FIG. 41 after interaction with a user;

FIG. 44 is a representation of a 3D model pane of the patient overview screen shown in FIG. 39 after interaction with a user;

FIG. 45 is a representation of a patient analysis overview pane of the patient overview screen shown in FIG. 39 after interaction with a user;

FIG. 46 is a representation of the patient analysis overview pane shown in FIG. 45 after the maximum stenosis level of a stenosis lesion has been changed by a user; and

FIG. 47 is a diagrammatic representation of the patient overview screen shown in FIG. 39 after selection of a measurements toggle button.

### DETAILED DESCRIPTION OF EMBODIMENTS

The present disclosure relates to a coronary artery disease (CAD) analysis system that is arranged to identify CAD, in the present example using coronary computed tomography (CT) data, communicate patient CAD related information to a user and facilitate interaction with the user, for example so as to receive instructions from the user, information from the user and/or edits from the user that aim to improve the accuracy of the CAD results presented to the user. In this example, the system is able to determine a CAC score for a patient, detect the presence and severity of individual stenosis lesions, and identify early stages of coronary artery disease and/or high patient risk by identifying vulnerable plaques (sometimes referred to in this specification as ‘plaque features’) including spotty calcification, low attenuation plaques and positive remodelling of the vessel walls.

However, while the present embodiment is described in relation to a CAD analysis system that both determines a CAC score and analyses coronary arteries for presence of stenosis lesions and/or vulnerable plaques, it will be understood that variations within the context of the present invention are envisaged.

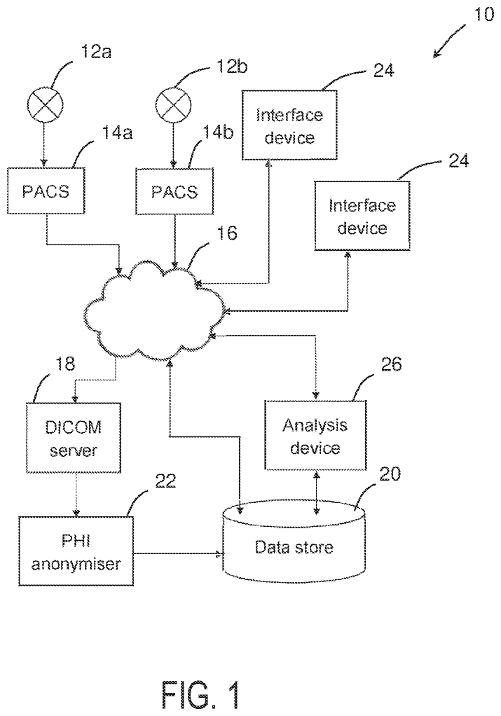

Referring to the drawings, FIG. 1 shows a schematic block diagram of a coronary artery disease (CAD) analysis system 10 according to an embodiment of the invention.

In this example, the system 10 is arranged to interact with multiple providers of cardiac computed tomography (CT) data, represented in FIG. 1 by CT scanning devices 12a, 12b and associated Picture Archiving and Communication Systems (PACS) 14a, 14b. Each PACS system 14a, 14b is arranged to manage capture and storage of medical image data produced by a CT scanning device 12a, 12b, and communication of the medical image data to a medical image data server 18, in this example disposed remotely of the CT service providers, and accessible through a wide area network such as the Internet 16. In this example, the medical image data server 18 is a Digital Imaging and Communications in Medicine (DICOM) server, although it will be understood that any suitable device for receiving and managing storage of received CT image data is envisaged.

The DICOM server 18 is arranged to store received CT image data in a data storage device 20 that may include one or more databases. In this example, the system 10 also includes a personal health information (PHI) anonymiser 22 that may be a separate component or a component incorporated into the DICOM server 18. The PHI anonymiser 22 is arranged to encrypt patient specific meta data (typically including name, date of birth and a unique ID number) in the received CT image data before the CT image data is stored in the data storage device 20. In this way, the patient specific meta data is still associated with the CT image data, but is only accessible by authorised people, for example using login and password data.

In the context of the invention, the CT image data may be derived from contrast and/or non-contrast CT scans.

The system 10 is arranged to enable multiple authorised users to interact with the system 10, for example by providing each authorised user with an interface device 24. Each interface device 24 may include any suitable computing device, such as a personal computer, laptop computer, tablet computer or mobile computing device.

The system 10 also includes a coronary artery disease (CAD) analysis device 26 in communication with the data storage device 20 and arranged to analyse CT image data stored in the data storage device 20 and produce analysis information relevant to prediction or assessment of coronary artery disease in the CT image data, either automatically or in response to user input.

The system 10 may be arranged to facilitate access using the interface device 24 in any suitable way. For example, the system 10 may be configured such that the CAD analysis device 26 is accessible through a web browser on the interface device 24, wherein all or most processing activity occurs remotely of the interface device 24, or the system 10 may be configured such that at least some processing activity occurs at the interface device 24, for example by providing the interface device 24 with at least one software application that implements at least some processing activity on the CT data stored at the data storage device 20.

In an alternative example, instead of providing a distributed system wherein CT data received from patients is stored remotely at a network accessible location, one or more components of the system 10 may be disposed at the same location as the interface device 24 and/or the CT device 12a, 12b such that most or all processing activity and/or storage of the CT data occurs at the same location.

In this example, the data stored at the data storage device 20 may also be accessible by an interface device 24 directly, for example so that a user at the interface device 24 can view raw CT data.

Using the interface device 24, a user is able to instigate analysis and/or view the results of analysis of CT data stored at the data storage device 20. During analysis, the CAD analysis device 26 extracts relevant CT data from the data storage device 20 and carries out analysis processes on the CT data in order to predict, identify, quantify and/or characterise coronary artery disease in the CT image data, either automatically or in response to use input.

A user interacts with the system 10 using a user interface 53 that communicates patient coronary artery disease related information to the user and facilitates reception of instructions and/or information from the user, for example relating to desired analysis information sought by the user, or relating to edits to parameters of the analysis carried out by the system 10 or edits to analysis information communicated to the user; and/or that facilitates reception of information from the user that supplements the analysis information generated by the system 10.

The user interface 53 is displayed on a screen of the interface device 24, presents information to a user and facilitates interaction with the user in a convenient, concise, intuitive and user-friendly way. In this way, the user is provided with an interface that enables the user to quickly ascertain relevant patient CAD-related information and thereby make determinations as to CAD risk, CAD existence and appropriate steps for mitigation and/or treatment.

In the present example, the system 10 is arranged to generate a CAC score by using machine learning techniques and radiomics, which enables enough information to be extracted from a non-contrast CT scan to correctly identify coronary calcifications and the artery they pertain to, without the need for contrast enhancement of the arteries or manual guidance. The system 10 may use machine learning to determine the most likely classification of every voxel in the CT scan, and machine learning to identify non-coronary artery features, which can then be used to remove or avoid misclassifications of components as calcified coronary artery components.

The system 10 is also arranged to use machine learning to identify, quantify and characterise coronary artery disease by detecting and tracking coronary artery centrelines, estimating the location of inner and outer walls of coronary arteries based on the centrelines and using machine learning, and determining the extent and characteristics of any identified disease using the estimated inner and outer walls together with an analysis of the composition and spatial characteristics of identified gaps between the inner and outer walls.

However, it will be understood that other methodologies are envisaged for determining a CAC score and/or analysing coronary arteries for risk or presence of CAD.

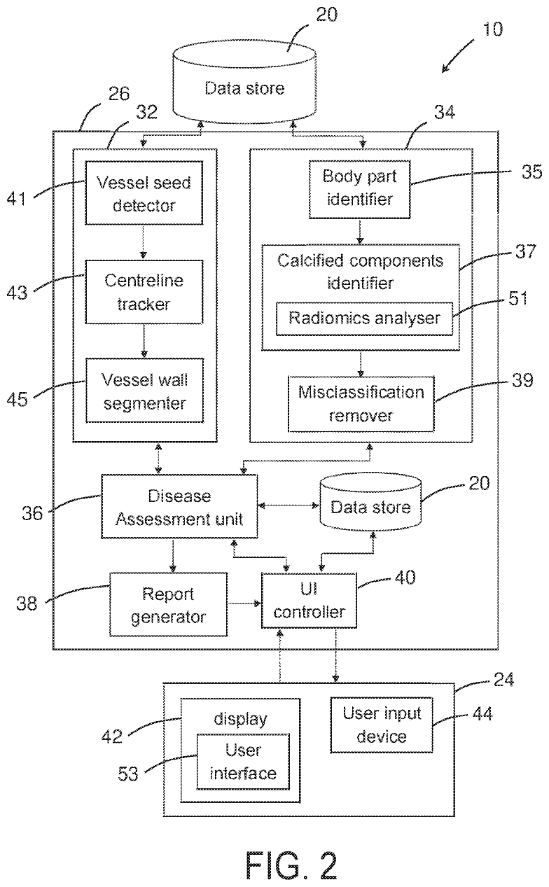

The CAD analysis device 26 is shown in more detail in FIG. 2.

The CAD analysis device 26 includes a coronary artery analysis component 32 arranged to analyse coronary arteries in contrast CT scan data based on segmentation of inner and outer walls of the coronary arteries, and a calcium score determining component 34 arranged to determine a calcium score based on non-contrast CT scan data.

The CAD analysis device 26 also includes a disease assessment unit 36 arranged to:
assess different types of disease including stenosis and presence of vulnerable plaques, including calcified, mixed or non-calcified plaques, based on the spatial characteristics and Hounsfield Unit values of gaps in the vessel walls; and
determine risk of coronary artery disease using the determined CAC score.

The determinations made by the disease assessment unit 36 are used by a report generator 38 to produce textual and/or numerical information indicative of the analysis carried out on a patient using the contrast and/or non-contrast CT scan data. At least some of the textual and/or numerical information is communicated to a user through the user interface 53.

In this example, the coronary artery analysis component 32 relies on segmentation of inner and outer walls of the coronary arteries and the information produced by this is used to detect and assess the disease burden in the scan. In order to accurately segment the vessel walls, centrelines of the coronary arteries are first determined by identifying a plurality of seed points on each centreline corresponding to voxels within the CT volume that are likely to be located on a centreline of a coronary artery. To facilitate this process, a contrast agent is injected into the blood stream to increase contrast and in this example increase a Hounsfield Unit (HU) value of the coronary arteries compared to the surrounding tissue.

The coronary artery analysis component 32 identifies vessel seed points using a vessel seed detector 41 that in this example uses multiscale filtering and supervised machine learning to detect seed points. In this example a volumetric convolutional neural network (CNN) is used that is trained using ground truth data indicative of a sufficient number of example coronary artery centrelines.

The vessel seed detector 41 identifies a set of predicted seed points present in a sample of CT data using machine learning, and selects candidate seed points from the set of predicted seed points that are to form the basis of centreline tracking and thereby prediction of the centrelines of the coronary arteries. The candidate vessel seed points are determined from the set of seed points based on one or more defined constraints, such as seed points that have a radiodensity value, such as a Hounsfield Unit (HU) value, above a defined amount, or a defined number of seed points above a defined HU threshold, such as a defined number of seed points that have the highest HU values. In one example, the candidate vessel seed points that have a HU value between 100 and 600 are selected as candidate seed points.

A centreline tracker 43 then considers the determined candidate seed points and predicts from an instant seed point the most probable direction of the next seed point on the coronary artery in three dimensional space using machine learning, and in this way vessel centreline seed points are identified that are likely to lie on the currently considered coronary artery. In this example, the centreline tracking process starts at a predicted seedpoint located at an endmost location on an artery centreline. The candidate seed points identified in this way as located on a coronary artery centreline are connected together so as to define a complete coronary artery.

The centreline tracker 43 is arranged to detect the four main coronary arteries first—the Left Main (LM), Left Anterior Descending (LAD), Left Circumflex (LCX) and the Right Coronary Artery (RCA), then after the main coronary arteries have been detected, branches on the primary coronary arteries are detected that were not initially identified as viable centrelines.

The centreline tracker 43 examines the HU values perpendicular to the centreline direction of a vessel, and estimates the approximate radius of the vessel by finding the boundary of the coronary artery based on the HU value, since the HU value decreases significantly outside of the vessel wall. Once the boundary has been located on each side of the centreline, the vessel's diameter can be measured.

Branches are detected based on the rate of change in measured diameter of the vessel along the length of a centreline. For example, if the measured diameter of the vessel increases by more than 10% along the centreline, then decreases back to its original size it is marked as a detected branch, noting that coronary vessels naturally decrease in size from a proximal to a distal location. At the coronary ostia, vessels may have a diameter of about 4 mm, whilst at a distal location the vessel diameter typically reduces to less than 1 mm. The branch detector therefore examines the rate of change of the estimated diameter to detect points along the centreline from which another coronary artery is branching.

The coronary artery analysis component 32 may then attach semantically meaningful labels to the tracked artery centrelines, for example using machine learning, so that clinicians can more easily identify the vessels.

In this example, the coronary artery analysis component 32 is also arranged to improve the reliability of the centreline tracking process by facilitating reconfiguration of the vessel seed detector 41 if the analysis carried out by the centreline tracker 43 is incorrect or incomplete, for example because the vessel seed detector 41 has generated too many or insufficient seed points. After the detected coronary arteries have been labelled by the centreline labeller, the parameters of the vessel seed detector 41 may be reconfigured if a determination is made that the identified vessels are incorrect or incomplete, for example if the initial vessel seed detector configuration failed to detect a major coronary artery, such as the RCA. For example, this may be achieved by lowering the constraint applied by the vessel seed detector 41 so that more candidate vessel seed points are produced, thereby increasing the probability of detecting the vessel in a subsequent iteration.

After all desired coronary arteries have been satisfactorily tracked and labelled, a vessel wall segmenter 45 uses the tracked centrelines to analyse the CT data associated with the coronary arteries, in particular to carry out an inner and outer vessel wall segmentation process.

The vessel wall segmenter 45 uses a machine learning component to produce inner and outer wall lumen masks that can then be used to identify coronary artery disease associated with the presence of calcified and non-calcified plaques. In this example, the machine learning component is a supervised volumetric convolutional neural network (CNN) that is trained using ground truth training data indicative of a sufficient number of example transverse coronary artery image slices, in this example image slices that are perpendicular to and intersecting with the artery centrelines. The training data in this example includes inner and outer artery walls and relevant imaging artefacts that have been annotated by medical experts, and covers a wide range of examples of different coronary vessels with varying degrees of disease and including various typical imaging artifacts indicative of abnormalities, such as vessel bulging.

It will be understood that after completion of coronary artery wall segmentation, the system has sufficient data to define the inner and outer vessel wall configurations of the detected coronary arteries. Using this data, it is possible to determine the presence of disease by analysing voxels associated with gap regions between the inner and outer vessel walls.

The coronary artery analysis component 32 is arranged to identify individual stenosis lesions 90 on the coronary arteries and classify the severity of each individual stenosis lesion 90.

Presence of individual stenosis lesions 90 and stenosis severity is determined by identifying start and end slices of the stenosis lesion, then categorising all slices between the start and end slices as belonging to the stenosis lesion. In this example, a start stenosis lesion slice is identified by reference to the lumen cross-sectional area and a corresponding normal cross-sectional area. For example, if the lumen cross-sectional area of a coronary artery is less than 99% of a corresponding normal cross-sectional area, the slice may be categorised as a start stenosis lesion slice, with the stenosis classification of the slice being defined according to the percentage reduction in cross-sectional area. Subsequent slices that also fall within the same stenosis classification by reference to the lumen cross-sectional area are also identified as part of the stenosis lesion 90 until an end stenosis lesion slice is identified. At the end stenosis lesion slice, the next slice does not have a lumen cross-sectional area that is within the same stenosis lesion classification.

In this way, the coronary artery analysis component 32 is able to separately identify multiple individual stenosis lesions on a coronary artery. In doing so, it becomes possible to display individual stenosis lesions to a user and to communicate to the user the characteristics of each individual stenosis lesion, for example using colour coding.

In this example, the calcium score determining component 34 includes a body part identifier 35 for identifying one or more non-coronary artery body part components in the cardiac non-contrast CT data, a calcified components identifier 37 for identifying calcified components in the cardiac non-contrast CT data based on a determined Hounsfield Unit (HU) value, and a misclassification remover 39 that uses the information from the body part identifier 35 to remove calcified volumes from consideration.

In this example, the body part identifier 35 is arranged to predict using machine learning whether each voxel in received patient cardiac non-contrast CT data is part of a non-coronary artery body part, such as an ascending or descending aorta of the patient, and to use a connected component technique to identify neighbouring voxels that belong to the same component. The body part identifier 35 produces a machine learning voxel mask that can be used to remove from consideration calcifications present on non-coronary artery body parts.

The calcified components identifier 37 is arranged to use a connected component technique to identify neighbouring voxels that belong to the same calcified component, and a radiomics analyser 51 to analyse the identified calcified components to obtain a set of characteristics for each component.

In the field of medicine, radiomics is used to extract information from radiographic medical images. The present inventors have realised that such radiomic features have the potential to be used in a machine learning system to identify and locate coronary artery calcifications. By analysing each candidate calcification component using a radiomics engine, radiomics characteristics, for example describing the relative position, shape, size, density and/or texture of the components are obtained, and these characteristics are chosen to provide a rich description of the components that can be used by machine learning systems to learn to distinguish non-coronary artery calcifications, such as bone, from coronary artery calcifications as well as the specific artery in which the calcifications are located. Prior to training, radiomic feature selection is performed by a principal component analysis (PCA) and variance thresholding. PCA is used to automatically determine which features provide the most discriminative power for the machine learning system. This approach provides additional benefits over the traditional prior art approach of hand-crafting specific features. A deep learning model may also look at image patches of raw CT data around each component in order to provide greater context.

In addition to the radiomic characteristic information, other information that is capable of assisting identification and classification of coronary artery calcifications may be used. For example, raw CT scan image patch information indicative of a region around each candidate calcification may be input to classifiers or to an additional machine learning system. Such image patches are capable of providing useful contextual information for each calcification.

In an example implementation, the component characteristics are input into a plurality of trained machine learning classifiers that have been trained to detect the locations of the components based on the characteristics. Alternatively, the component characteristics are used as inputs, for example with raw image data, to a trained deep learning model which predicts the location of the components based on the characteristics.

The predicted candidate calcifications produced by the trained machine learning classifiers are cross-checked against the body part information and any candidate calcifications that are considered to relate to noise, or to be present on the non-coronary artery body part(s), are removed.

The CAD analysis system uses the disease assessment unit 36 to make determinations based on the results of the CAD analysis carried out by the coronary artery analysis component 32 and the calcium score determining component 34. The determinations may be made and/or communicated to a user automatically or may be made and/or communicated to the user in response to user input. In this example, the determinations include stenosis detection and categorisation, CAC score calculation and vulnerable plaque detection and characterisation. In particular, the disease assessment unit 36 uses the inner and outer wall segmentation data to determine the cross-sectional area defined by the inner wall, and based on this a stenosis condition is characterised with reference to a healthy state condition.

Vulnerable plaques (VP), also referred to as high-risk plaques, are an early indication of coronary artery disease for a patient. The disease assessment unit 86 detects several forms of VP using heuristic, rule-based analysis of the artery wall segmentation, in this example low attenuation plaque, spotty calcification and positive remodelling.

Low attenuation plaques are characterised by Hounsfield Unit (HU) values in the range −30 to 30 Hounsfield units, and therefore may be directly detected through analysis and thresholding of Hounsfield units.

A spotty calcification is defined as a relatively small calcification surrounded by non-calcified or mixed plaque. To detect spotty calcification, the disease assessment unit 36 initially determines voxels that are predicted to be associated with calcified plaques in the determined disease region between the inner and outer artery wall, for example by filtering using a defined radiodensity measure, such as a Hounsfield Unit (HU) value greater than 350. Related voxels are then associated together as calcified volumes. Spotty calcifications are characterised as being smaller than 3 mm in diameter. Non-calcified/mixed plaque is used to determine whether the voxels surrounding the identified spotty calcifications have HU values consistent with non-calcified or mixed plaques.

Positive remodelling is characterised by an expansion of the outer vessel wall to compensate for the disease build up between the inner and outer wall. The disease assessment unit 36 is arranged to detect this using an inner/outer wall gap determiner that determines whether the gap between the inner and outer artery wall has increased beyond a defined amount, for example 10% beyond a normal vessel gap. The radiodensity of voxels in the gap are consistent with non-calcified plaque, for example by determining the HU values of the voxels in the gap.

The CAD analysis system 30 also includes a UI controller 40 arranged to package the information produced by the disease assessment unit 36 and the report generator 38, and any required data from the data store 20, into a user interface 53 displayed on a suitable display 42, the user interface 53 configured such that patient CAD related information is communicated to a user in a way that enables the user to quickly and intuitively obtain relevant CAD information for a patient, and that enables a user to provide inputs using an input device 44, for example in order to edit analysis parameters and/or add or amend analysis information. For example, the UI controller 40 is arranged to produce a 3D model of the detected coronary arteries of a patient that have been derived from the CT data, produce representations of transverse slices of the coronary arteries with superimposed segmented inner and outer wall annotations, and provide user friendly tools that enable the user to quickly identify locations and extent of CAD or factors that indicate a risk of CAD.

In order to access the user interface 53, a user accesses the analysis device 26, for example using an interface device 24 that may be a personal computer, laptop computer, tablet computer or smartphone, and enters login information. The user interface 53 is displayed after successful login.

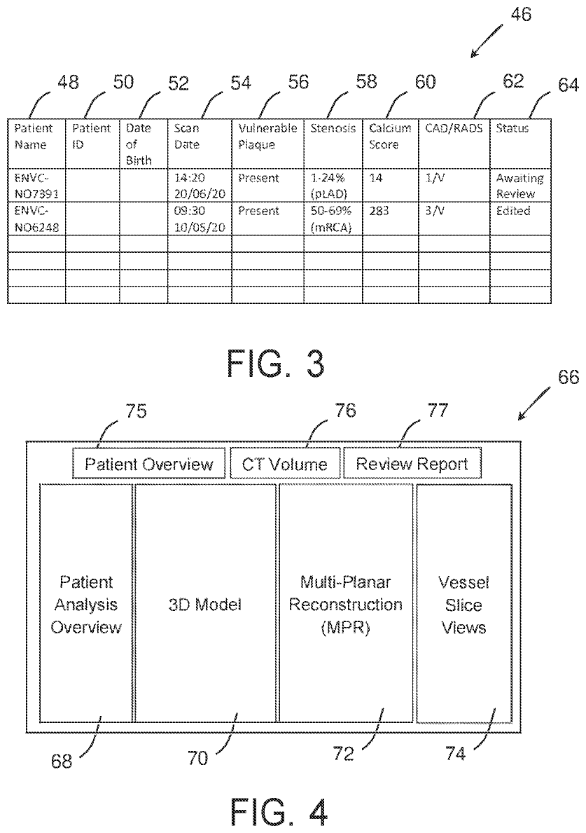

Example screens of a user interface 53 of an embodiment of a CAD analysis system displayed to a user after successful user authentication are shown in FIGS. 3 to 38.

In this embodiment, the CAD analysis system is arranged to automatically make disease assessment determinations, for example in relation to presence and severity of stenosis, calcium score calculation, and vulnerable plaque detection and characterisation, and to automatically display information indicative of the determinations on the user interface 53.

After successful login, a scan menu 46 as shown in FIG. 3 is displayed. The scan menu 46 includes a list of all scan datasets that are accessible by the user. In this example, for each scan dataset, the following information may be included:
patient name 48;
patient ID 50;
patient date of birth 52;
scan date 54;
an indication 56 as to whether vulnerable plaque is considered to be present;
an indication 58 as to whether stenosis is considered to be present;
a calcium (Agatston) score 60;
a CAD-RADS classification 62; and
a dataset status 62 indicating whether the status of the dataset is awaiting review, edited, ready for approval or approved.

However, it will be understood that any suitable information may be included in the scan menu 46.

Using the scan menu 46, a user is able to select a dataset to review and/or edit, for example using a mouse or by touching the relevant dataset row if a touch screen is present. Selection of a dataset row causes a patent overview screen 66 to be displayed, as shown in FIG. 4.

The patient overview screen 66 includes a patient analysis overview pane 68 that displays a summary of CAD results in data summary and textual form, a 3D model pane 70 that displays a 3D structural model of coronary arteries identified in the dataset, a multiplanar reconstruction (MPR) pane 72 that displays an MPR view of the CT data, and a vessel slice pane 74 that displays one or more views of axial slices taken through a selected coronary artery.

However, it will be understood that the patient overview screen 66 may include different or additional view panes that may be customisable by a user or that may change according to the functionality selected by a user. For example, the patient overview screen 66 may include any of the following view panes:
a patient analysis overview pane 68;
a 3D model pane 70;
a multiplanar reconstruction (MPR) pane 72;
a vessel slice pane 74;
one or more CPR panes;
one or more SPR panes;
one or more non-contrast view panes;
one or more CT volume panes; and
axial, sagittal and/or coronal view panes.

A multiplanar reconstruction (or reformation) (MPR) is obtained by extracting data from acquired images, in this example in multiple axial planes so that a selected vessel that extends across multiple planes can be shown in a single view. A curved planar reformation (CPR) and/or a straightened planar reformation (SPR) can be produced so as to display a two-dimensional image of a vessel that spans multiple different planes. The acquired data can be converted to non-axial planes such as coronal or sagittal.

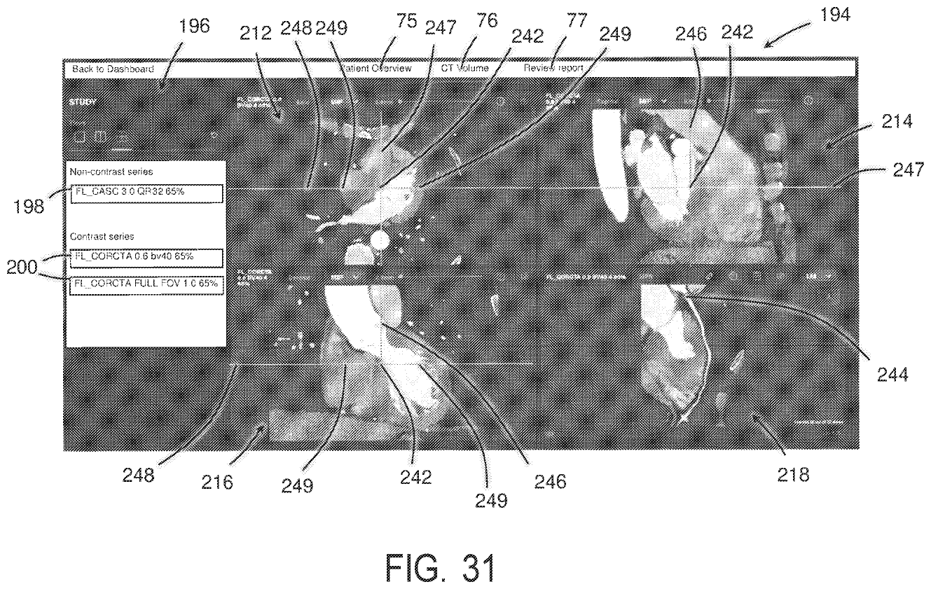

The patient overview screen 66 also includes screen selection buttons, including a patient overview button 75, a CT volume button 76 and a review report button 77, that are usable to switch between the patient overview screen 66, a CT volume screen 194 shown in FIG. 22, and a report screen 250 shown in FIG. 31.

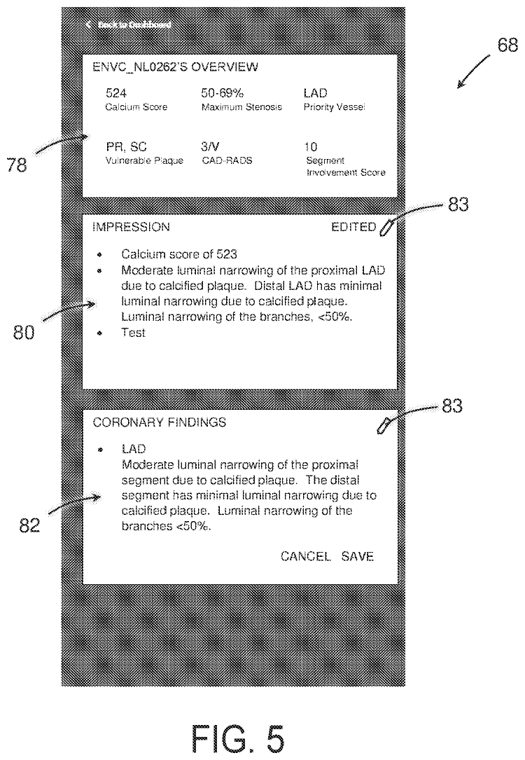

An example representation of the patient analysis overview pane 68 is shown in FIG. 5. The example patient analysis overview pane 68 includes a results summary section 78 that in this example includes the following information:
a calcium (Agatston) score;
a maximum stenosis level that indicates the highest level of stenosis determined in the dataset;
an indication of the priority vessel, that is, the coronary artery that includes the most significant stenosis lesion;
an indication of the vulnerable plaque (if any) present in the dataset;
the relevant CAD-RADS classification; and
a segment involvement score that indicates how many stenosis lesions are present in the dataset.

It will be understood that the calcium score shown on the patient analysis overview pane 68 represents the total calcium determined to be present in the coronary arteries.

In the present example, the following stenosis levels are used:
0%—no evidence of stenosis;
1-24%—minimal stenosis;
25-49%—mild stenosis;
50-69%—moderate stenosis;
70-99%—severe stenosis; and
100%—occluded.

In the present example, the following plaque types are used:
none;
non-calcified;
mixed; and
calcified.

In the present example, the following vulnerable plaque (VP) characterisations are used:
LAP—low attenuation plaque;
PR—positive remodelling; and
SC—spotty calcification.

In the present example, the CAD-RADS classification uses the following notation:
CAD-RADS 0: 0%/absence of coronary artery disease
CAD-RADS 1: 1-24%/minimal nonobstructive coronary artery disease or plaque with no stenosis (positive remodelling)
CAD-RADS 2: 25-49%/mild nonobstructive coronary artery disease
CAD-RADS 3: 50-69%/moderate stenosis
CAD-RADS 4: severe stenosis
   CAD-RADS 4A: 70-99% stenosis
   CAD-RADS 4B: left main >50% stenosis or three-vessel obstructive (≥70% stenosis) disease
CAD-RADS 5: 100%/total occlusion
CAD-RADS N: nondiagnostic study

The following modifiers are also used:
modifier N: nondiagnostic
modifier S: stent
modifier G: graft
modifier V: vulnerability

The example patient analysis overview pane 68 also includes an overall impression section 80 that provides in words a summary of the dataset analysis, and in this example the overall impression section 80 indicates that the total coronary artery calcium score is 523 for the patient associated with the dataset, modified luminal narrowing of the proximal LAD artery due to calcified plaque exists, minimal luminal narrowing of the distal LAD artery due to calcified plaque exists, and luminal narrowing of the arterial branches exists (<50%).

The example patient analysis overview pane 68 also includes a vessel findings section 82 that provides in words a summary of each coronary artery that has a finding of significance.

The information in the overall impression section 80 and the vessel findings section 82 may be edited from the patient analysis overview pane 68 using edit links 83.

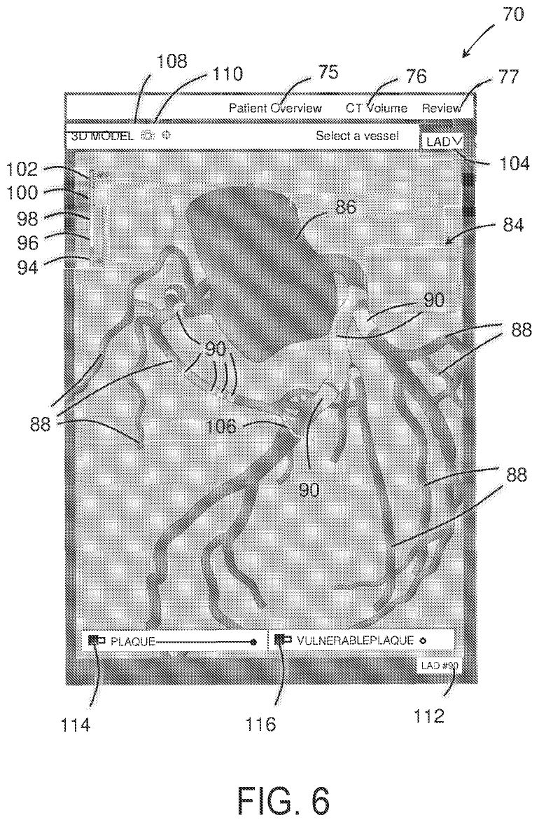

An example representation of the 3D model pane 70 is shown in FIG. 6. The example 3D model pane 70 includes a 3D model 84 of the coronary arteries of the patient associated with the dataset. The 3D model 84 serves as a model of the patient's coronary arteries and is produced by the UI controller 40 using data produced by the analysis device 26, in particular, in this example, using the segmented walls of the coronary arteries.

The 3D model 84 includes models of a portion of the patient aorta 86 and coronary arteries 88, and also identified coronary artery stenosis lesions 90 and the respective locations on the coronary arteries of the stenosis lesions 90. Each stenosis lesion is represented differently according to the respective stenosis lesion characteristics, and in this example colour is used to indicate presence of stenosis and stenosis severity.

The 3D model pane 70 includes a stenosis level colour key 92 to provide an indication of stenosis severity according to colour. In this example, the following colours are used to indicate stenosis:
0% stenosis—grey 94;
1-24% minimal stenosis—white 96;
25-49% mild stenosis—yellow 98;
50-69% moderate stenosis—orange 100; and
70-100% severe stenosis/occluded—red 102.

A user is able to select a vessel either by directly selecting the vessel on the 3D model 84, for example using a mouse or touch screen, or by selecting the vessel using a vessel drop down box 104. After selection of a vessel, a vessel slice is marked on the 3D model 84 using a vessel slice identifier 106, in this example in the form of a square frame.

The 3D model pane 70 also includes a snapshot selection button 108 that when selected causes a snapshot of the currently displayed 3D model 84 to be captured, the snapshot usable to add annotations as discussed in more detail below.

The 3D model pane 70 also includes a most significant stenosis lesion button 110 that when selected causes the vessel slice identifier 106 to be disposed on the stenosis lesion 90 with the most significant stenosis level.

The 3D model pane 70 also includes a calcified plaque toggle button 114 usable to show or hide calcified plaque on the 3D model 84. As shown in FIG. 20, when the calcified plaque toggle button 114 is toggled to an ON position, determined calcified volumes 192 (in this example shown in white) are shown.

It will be understood that in this example data indicative of the calcified volumes 192 is obtained using the calcium score determining component 34.

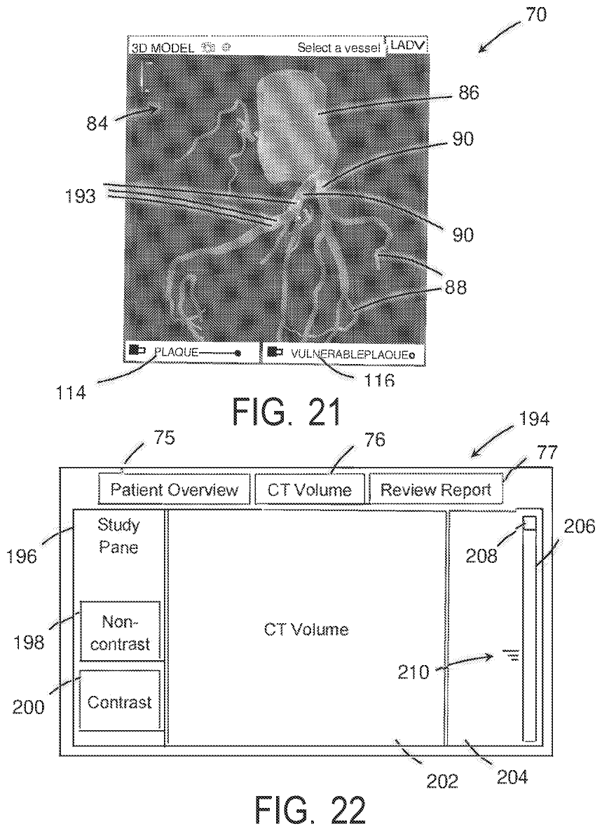

The 3D model pane 70 also includes a vulnerable plaque toggle button 114 usable to show or hide vulnerable plaque on the 3D model 84. As shown in FIG. 21, when the vulnerable plaque toggle button 116 is toggled to an ON position, locations 193 of vulnerable plaque are indicated on the 3D model 84 using dots (in this example shown in white).

It will be understood that in this example data indicative of the locations 193 of vulnerable plaque is obtained using the coronary artery analysis component 32.

In this example, a user is able to manipulate the 3D model 84 shown in the 3D model pane so as to change the displayed orientation of the 3D model 84, for example using a mouse. The orientation of the 3D model may be modifiable about 1, 2 or 3 mutually orthogonal axes.

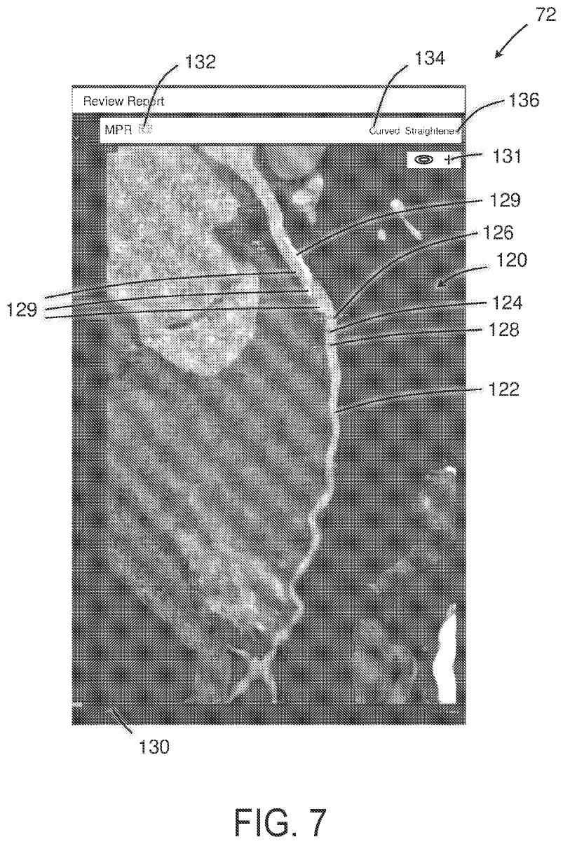

An example representation of the MPR pane 72 is shown in FIG. 7. The example MPR pane 72 shows an MPR representation 120 derived from the CT data obtained from a CT scanning device 12, and showing the coronary artery 122 selected on the 3D model pane 70. In this example, the LAD coronary artery is selected on the 3D model 84 shown in FIG. 6, so the LAD coronary artery is displayed on the MPR pane 72.

The coronary artery 122 shown on the MPR representation 120 includes a selected vessel slice identifier 124 that marks the vessel slice corresponding to the vessel slice marked by the vessel slice identifier 106 shown on the 3D model pane 70.

It will be understood that a vessel slice may be selected on the MPR representation 120 by the user instead of on the 3D model pane 70, and that this causes the vessel slice identifier 106 to move, if necessary, according to the location of the vessel slice identifier 124 shown on the MPR representation 120.

In this example, the MPR representation 120 also includes a proximal slice identifier 126 and a distal slice identifier 128 that are also selectable on the displayed coronary artery 122.

In this example, the MPR representation 120 also includes representations of calcified volumes 129 that are present on the displayed coronary artery 122.

As discussed below, the locations of the slice identifier 124, the proximal slice identifier 126 and the distal slice identifier 128 determine the axial slice views that are displayed in the vessel slice pane 74.

In this example, the MPR pane 72 also includes a view centreline button 130 that when selected causes a centreline 186 to be displayed on the selected coronary artery 122 shown in the MPR pane 72, as shown in FIG. 18, and an add centreline button 131 that when selected enables a user to add a new centreline, for example for a coronary artery that has not been detected by the analysis device 26.

In the present example, in order to add a new centreline, a user first selects the initial location of the new centreline on the displayed coronary artery 122 and subsequently selects one or more further representative locations for the new centreline. In response, the new centreline is displayed on the MPR representation 120.

In this example, after a new centreline has been added, the analysis device 26 analyses the new centreline to generate vessel wall segmentations and perform a disease assessment analysis based on the inner and outer wall segmentations in order to determine the presence of stenosis, plaque and/or vulnerable plaque.

In this way, in response to minimal interaction with the MPR representation by a user, the results produced by the CAD analysis system 10 can be improved to include a previously missed coronary artery.

The MPR pane 72 also includes a snapshot selection button 132 that when selected causes a snapshot of the currently displayed 3D model 84 to be captured, the snapshot usable to add annotations as discussed in more detail below.

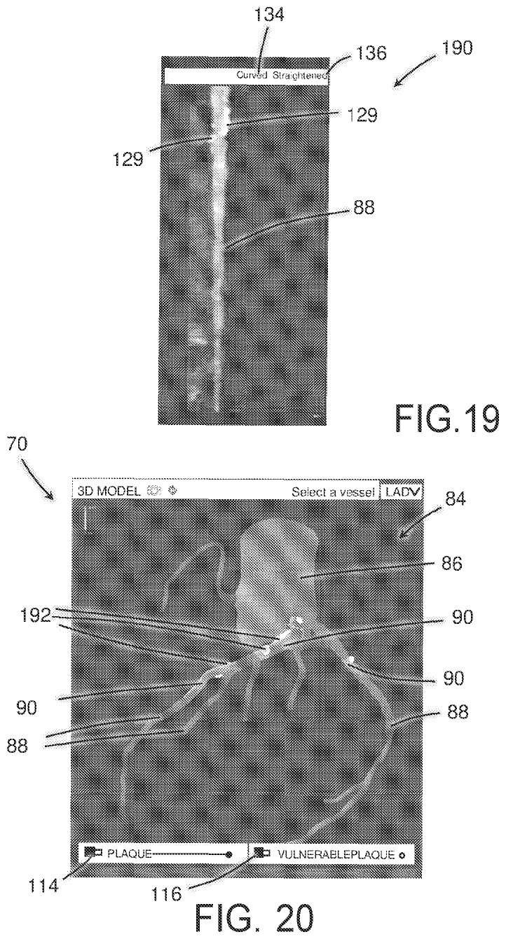

The MPR pane 72 also includes curved 134 and straightened 136 buttons that when selected cause a natural curved representation of the selected coronary artery to be displayed, as shown in FIG. 7, or a transformed straightened representation 190 to be displayed, as shown in FIG. 19.

The displayed coronary artery 122 may include a visual indication of stenosis lesions if a stenosis lesion is considered to be present. For example, a portion of the displayed coronary artery 122 corresponding to the location of a stenosis lesion may be displayed in a different colour, such as a colour corresponding to the stenosis severity used on the 3D model 84.

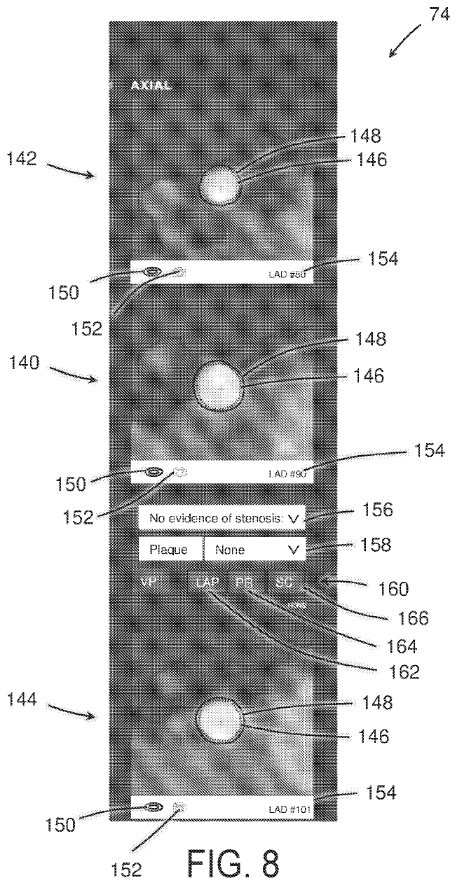

An example representation of the vessel slice pane 74 is shown in FIG. 8. The vessel slice pane 74 shows a representation 140 of a selected slice corresponding to the vessel slice identifiers 106, 124 shown on the 3D model pane 70 and the MPR pane 72, a representation 142 of a proximal slice corresponding to the proximal vessel slice identifier 126 shown on the MPR pane 72, and a representation 144 of a distal slice corresponding to the distal vessel slice identifier 128 shown on the MPR pane 72.

Each of the slice representations 140, 142, 144 includes an inner vessel wall annotation 146 and an outer vessel wall annotation 148 that are derived according to analysis carried out on the patient CT dataset by the coronary artery analysis component 32, in particular the machine learning assisted centreline tracking and wall segmentation components of the coronary artery analysis component 32.

Each of the slice representations 140, 142, 144 includes a wall annotation toggle button 150 that removes the wall annotations 146, 148 from display when toggled to an OFF position.

Each of the slice representations 140, 142, 144 also includes a snapshot selection button 152 that when selected causes a snapshot of the currently displayed slice representation 140, 142, 144 to be captured, the snapshot usable to add annotations as discussed in more detail below.

Each of the slice representations 140, 142, 144 also includes slice indicia 154 that identifies the particular slice of the selected coronary artery 122 with which the slice representation is associated. For example, in the example shown in FIG. 8, the representation 140 of the selected slice is associated with a 90^{th} slice of the selected coronary artery 122.

The representation 140 of the selected slice also includes a stenosis level box 156 that indicates the maximum stenosis level of the stenosis lesion with which the selected slice is associated, a plaque type box 158 that indicates the type of plaque present in the stenosis lesion, and vulnerable plaque labels 160 that indicate the type of vulnerable plaque present on the stenosis lesion. In the present example, the vulnerable plaque labels 160 include a low attenuation plaque label 162, a positive remodelling label 164 and a spotty calcification label 166.

In this example, the colour of the stenosis level box 156 is the same as the colour of the respective stenosis lesion 90 shown in the 3D model 84 so that the user can quickly identify the stenosis level of the stenosis lesion based on the colour of the stenosis level box 156.

The displayed slice representation 140, 142, 144 may include a visual indication of stenosis lesions if a stenosis lesion is considered to be present. For example, a slice representation 140, 142, 144 corresponding to the location of a stenosis lesion may be displayed in a different colour, such as a colour corresponding to the stenosis severity used on the 3D model 84.

The stenosis level indicated in the stenosis level box 156, the plaque classification indicated in the plaque type box 158, and the vulnerable plaque indicated by the vulnerable plaque labels 160 are determined according to analysis carried out on the patient CT dataset by the coronary artery analysis component 32.

As shown in FIG. 8, the axial slice representation 140 corresponding to the selected slice shown in FIGS. 6 and 7 and marked with the vessel slice indicator 106 is considered to have no evidence of stenosis, no plaque and no vulnerable plaque.

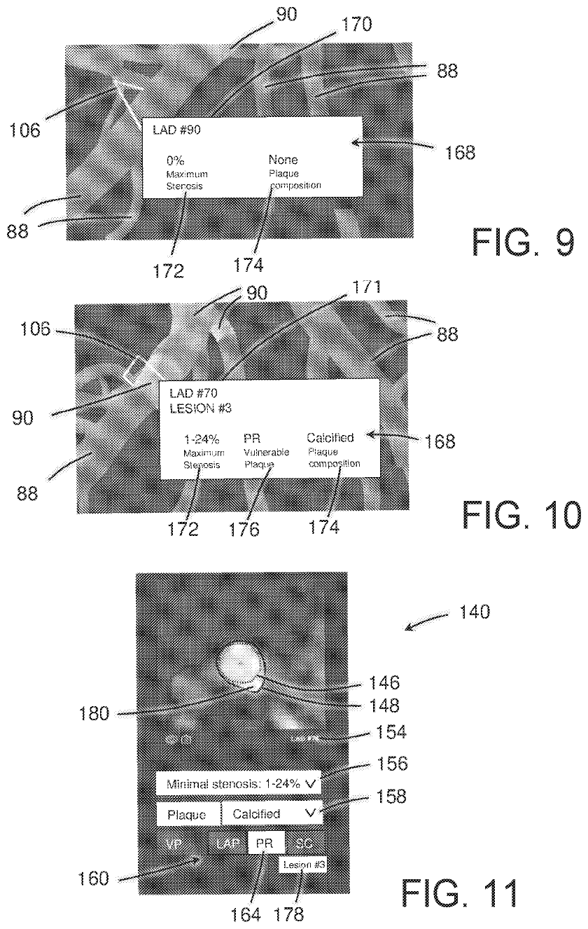

As shown in FIGS. 9 and 10, the stenosis, plaque and vulnerable plaque information associated with a stenosis lesion 90 may also be viewed on the 3D model 84, for example by hovering a mouse over a stenosis lesion 90 which causes a stenosis lesion information box 168 to be displayed. The stenosis lesion information box 168 may for example include slice indicia 170 that indicates the current slice corresponding to the selected location on a coronary artery 88, maximum stenosis indicia 172 that indicates the maximum stenosis level on the selected stenosis lesion 90, calcified plaque indicia 174 that indicates the type of calcified plaque, if any, present on the stenosis lesion 90, and vulnerable plaque indicia that indicates the type of vulnerable plaque, if any, present on the stenosis lesion 90.

FIG. 11 shows an axial slice representation 140 corresponding to the selected slice shown in FIG. 10 and marked with the vessel slice indicator 106. As shown, the axial slice representation 140 in FIG. 11 is considered be part of a stenosis lesion that has 1-24% stenosis, and as such the stenosis level box 156 is shown in the colour corresponding to a 1-24% stenosis level. The axial slice representation 140 in FIG. 11 is also considered to include calcified plaque and vulnerable plaque (positive remodelling).

As shown in FIG. 11, the axial slice representation 140 shows a calcified volume 180 between inner and outer vessel walls 146, 148, with the outer vessel wall deformed as a result. The displayed calcified volume may include a visual indication of plaque type, for example by assigning a different colour to each plaque type and displaying the calcified volume in a colour that corresponds to the determined plaque type.

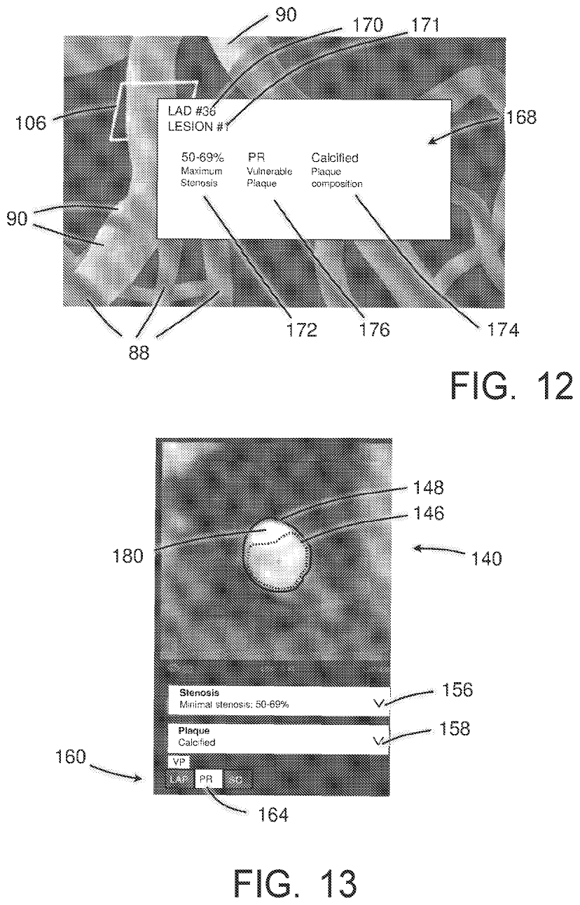

A further example slice, stenosis lesion 90 and associated axial slice representation 140 are shown in FIGS. 12 and 13. As shown in FIG. 13, the axial slice representation 140 shows a 50-69% stenosis level 156, calcified plaque 158 and positive remodelling 164.

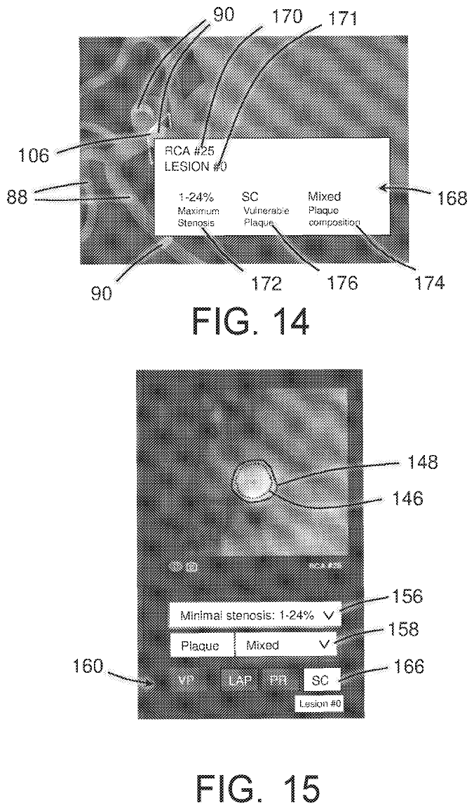

A further example slice, stenosis lesion 90 and associated axial slice representation 140 are shown in FIGS. 14 and 15. As shown in FIG. 15, the axial slice representation 140 shows a 1-24% stenosis level 156, mixed calcification 158 and spotty calcification 166.

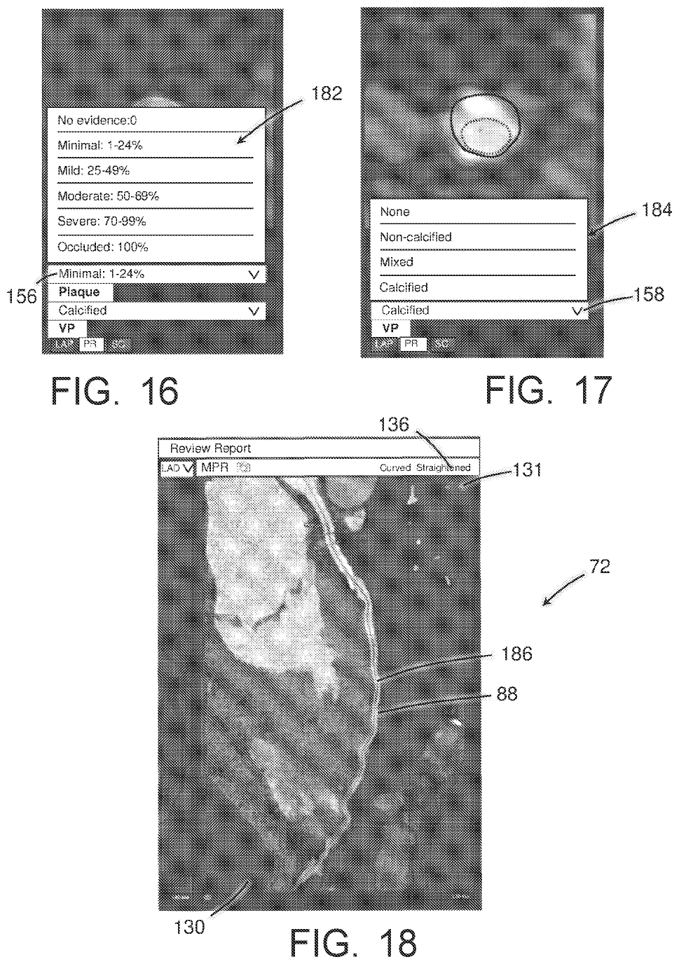

As shown in FIG. 16, if a user considers that the stenosis level determined by the system 10 for a stenosis lesion is incorrect, the user may edit the stenosis level using the stenosis level box 156 and a drop-down stenosis selection list 182. Similarly, as shown in FIG. 17, if a user considers that the plaque type determined by the system 10 for a stenosis lesion 90 is incorrect, the user may edit the plaque type using the plaque type box 156 and a drop-down calcification selection list 184.

Referring to the patient overview screen shown in FIG. 4, selection of the CT volume button 76 causes a CT volume screen 194 shown in FIG. 22 to be displayed.

The CT volume screen 194 includes a study pane 196 that includes tiles representing the CT scans available, in the present example a non-contrast tile 198 associated with a non-contrast scan for the dataset selected on the scan menu 46, and a contrast tile 200 associated with a contrast scan of the dataset, a CT volume pane 202 that shows a calcium CT volume, and a scroll bar pane 204 provided with a scroll bar 206, a position indicator 208 and calcium indicia 210.

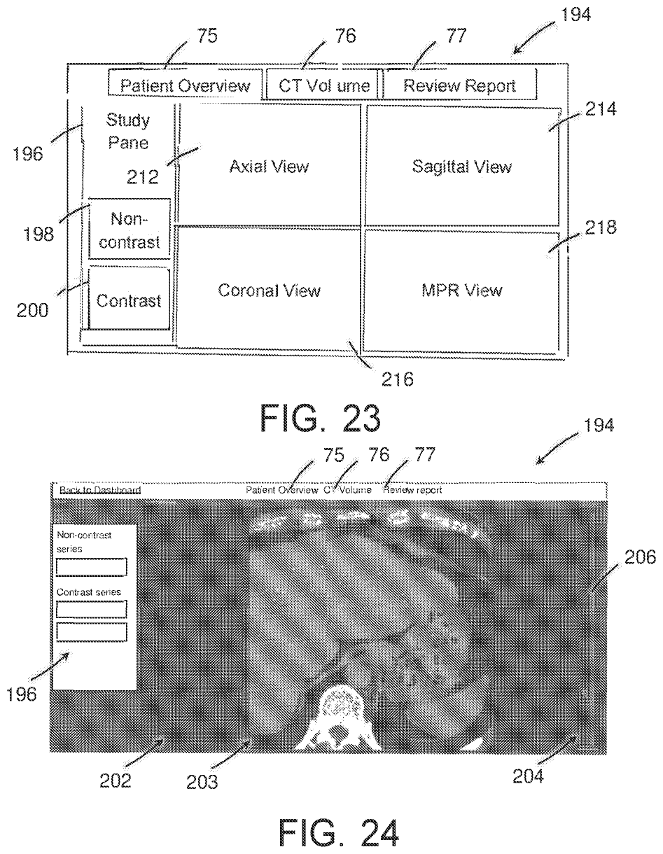

Selection of a contrast tile 198 causes a multi-view screen 195 to be displayed, as shown in FIG. 23. The multi-view screen includes axial view 212, sagittal view 214, coronal view 216 and MPR view 218 panes instead of the CT volume pane 202 and the scroll bar pane 204. The axial view pane 212 shows an axial representation of the CT volume, the sagittal view pane 214 shows a sagittal representation of the CT volume, the coronal view pane 216 shows a coronal representation of the CT volume, and the MPR view pane 218 shows an MPR representation of the CT volume. The axial representation, sagittal representation, coronal representation and MPR representation are synchronised such that as one of the representations is modified, the other representations are also modified so that a particular feature desired to be viewed is shown in multiple views.

The scroll bar 206 represents a set of axial slices of a CT volume and the position indicator 208 is used to select the axial CT volume slice 203 to be shown in the CT volume pane 202. The calcium indicia 210 shows the respective axial locations of calcified volumes in the CT volume.

An enlarged view of the scroll bar 206 is shown in FIG. 25. As shown, the calcium indicia 210 in this example includes a line 220 if the respective CT volume slice includes a coronary artery calcified volume, with characteristics of the line such as the colour of the line indicating the particular coronary artery on which the calcified volume is located, and the length of the line indicating the size of the calcification. As shown in FIG. 26, disposing the position indicator 208 adjacent a line 220 causes the associated axial CT slice representation 203 to be displayed in the CT volume pane 202, including the calcified volume 222 associated with the line 220 in a colour corresponding to the designated colour for the relevant coronary artery. Selection of a line 220, for example using a mouse or touch screen, may also cause the associated axial CT slice representation 203 to be displayed in the CT volume pane 202, together with the calcified volume 222 associated with the line 220.

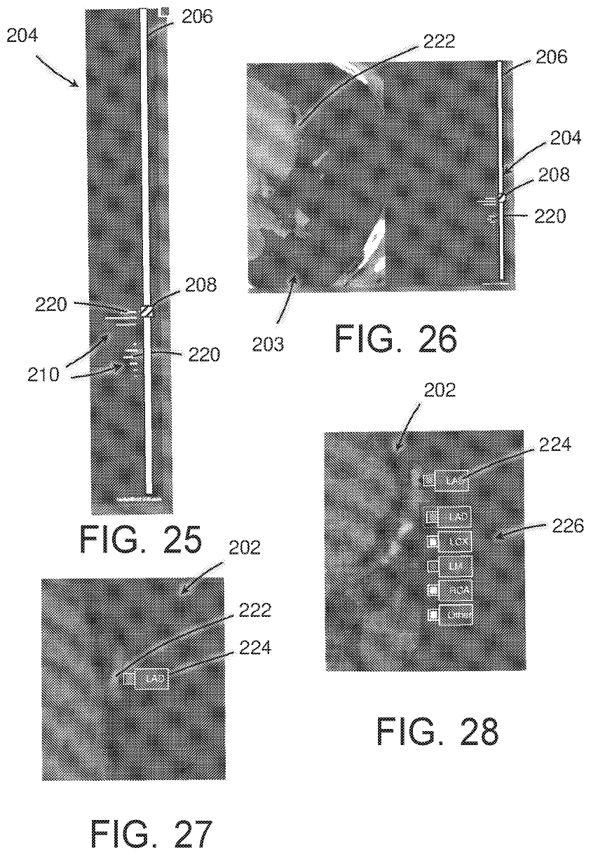

As shown in FIG. 27, a vessel label 224 may be displayed adjacent the calcified volume 222, for example in response to hovering a mouse over the calcified volume 222.

It will be understood that a user is able to use the scroll bar 26 to:
quickly receive an indication of the extent of coronary artery calcification by virtue of the number and distribution of lines 220;
quickly receive an indication of the respective axial locations of coronary artery calcified volumes for a patient based on the respective locations of the calcium indicia 210;
quickly identify the coronary arteries that include calcifications by virtue of the colour of the calcium indicia 210; and
determine the relative size of the calcifications by virtue of the size of the lines 220.

As shown in FIG. 28, the system 10 may be arranged to facilitate editing of the coronary artery allocated to the calcified volume 222 by the system 10, for example by selecting the displayed vessel label 224 and selecting a coronary artery from a vessel selection list 226.

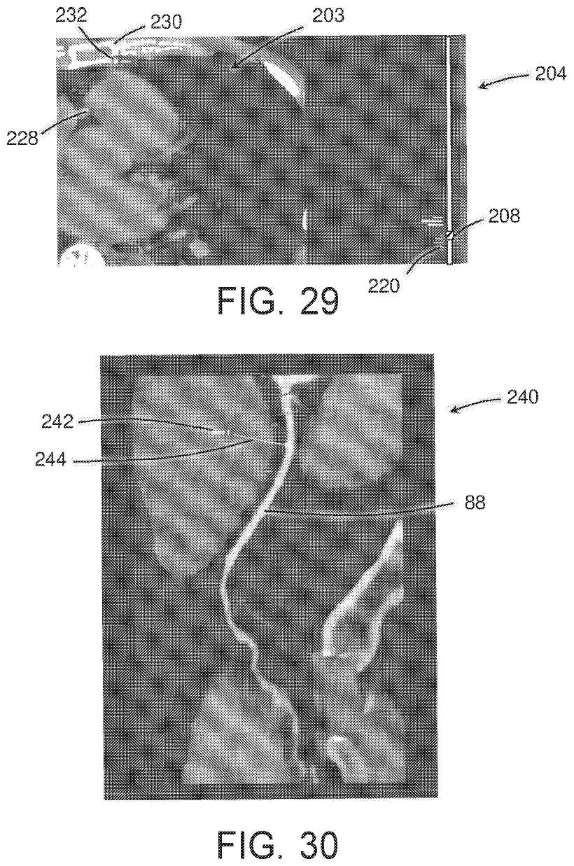

A further calcified volume 228 is shown in FIG. 29, the line 220 associated with the further calcified volume 228 and the further calcified volume 228 represented differently such as in a different colour to indicate that the further calcified volume is considered to be associated with a different coronary artery. As shown in FIG. 29, non-coronary artery calcium 230 may also be displayed, and a label such as ‘other’ may be displayed to indicate that the calcium is not associated with a coronary artery. The non-coronary artery calcium 230 may be displayed in a further different colour to the calcifications that are disposed on the coronary arteries.

An example multi-view screen 195 is shown in FIGS. 31 to 33, the multi-view screen 195 including an axial representation 234 of the CT volume in the axial view pane 212, a sagittal representation 236 of the CT volume in the sagittal view pane 214, a coronal representation 238 of the CT volume in the coronal view pane 216, and an MPR representation 240 of a selected vessel of the CT volume in the MPR view pane 218.

The MPR representation 240 includes a selected vessel slice indicator 244 that serves to indicate a selected location of the vessel displayed in the MPR view pane 218. The axial representation 234, sagittal representation 236 and coronal representation 238 are synchronised with the selected location of the vessel in that each of the axial, sagittal and coronal representations 234, 236, 238 show different views of voxels corresponding to the selected vessel location, the voxels indicated using a marker device 242, in this example a circle disposed centrally of the representation.

In this example, the marker device on each of the axial, sagittal and coronal representations 234, 236, 238 also represents a line normal to the displayed plane such that the axial representation 234 includes an axial normal line 246, the coronal representation 238 includes a coronal normal line 247, and the sagittal representation 236 includes a sagittal normal line 248. In this embodiment, the axial, coronal and sagittal normal lines 246, 247, 248 are represented differently, such as in different colours. For example, the axial normal line 246 may be represented in blue, the coronal normal line 247 may be represented in yellow and the sagittal normal line 248 may be represented in red. As shown in FIGS. 31 to 33, the marker device 242, in this example a circle, is represented in the colour of the associated line that is normal to the plane of the displayed axial, coronal or sagittal representation. It will be understood that the axial normal line 246 corresponds to a line extending centrally through a person from head to foot, the coronal normal line 247 corresponds to a line perpendicular to the axial normal line 246 and extending through a person from front to back, and the sagittal normal line 248 corresponds to a line perpendicular to the axial normal line 246 and the coronal normal line 247 and extending through a person from left to right.

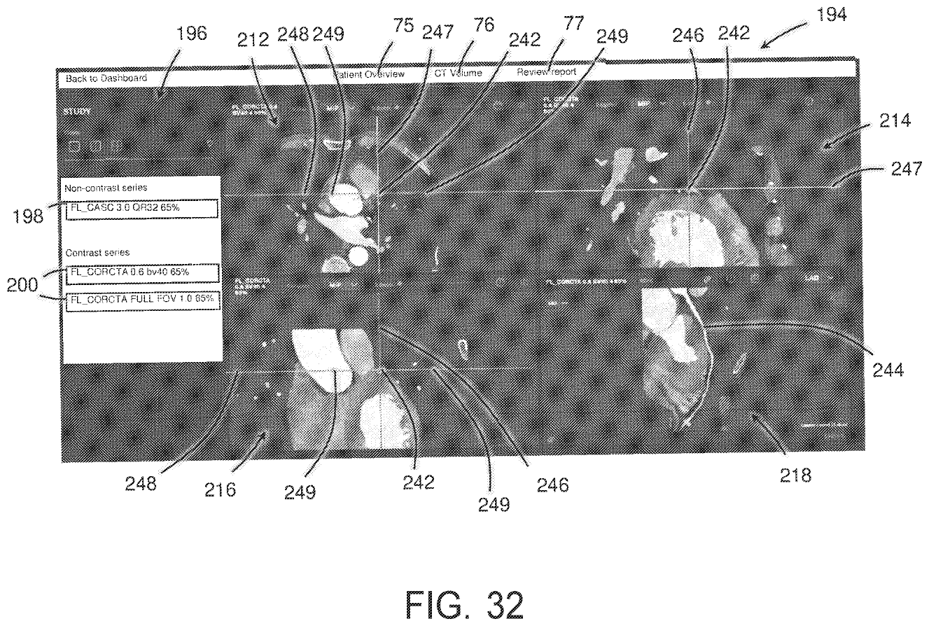

In this example, by interacting with the MPR view pane 218, a user is able to change the MPR view of the vessel, for example so as to rotate the vessel view. In this example, this can be achieved by clicking on a mouse and simultaneously moving the mouse left or right, although it will be understood that any suitable interface arrangement for achieving this is envisaged. A user is also able to move the selected vessel slice indicator 244 along the displayed vessel, which causes the marker device 242 to move and thereby the axial, coronal and sagittal representations to change in order to remain in synchronisation with the selected vessel location, as shown in FIG. 32.

In this example, by interacting with the axial view pane 212, the sagittal view pane 214, or the coronal view pane 216, a user is able to change the location of the view along the respective normal line. For example, clicking on a mouse with the mouse pointer located on the axial representation 234 and simultaneously moving the mouse up or down causes a different location along the axial normal line 246 to be selected and therefore a different axial representation corresponding to the different location along the axial normal line 246 to be displayed. Similarly, for example, clicking on a mouse with the mouse pointer located on the coronal representation 238 and simultaneously moving the mouse up or down causes a different location along the coronal normal line 246 to be selected and therefore a different coronal representation corresponding to the different location along the coronal normal line 247 to be displayed.

In addition, in this example, the system is arranged such that a user is able to interact with at least one of the axial, sagittal and coronal representations 234, 236, 238 to change the orientation of one or more of the axial, coronal or sagittal normal lines 246, 247, 248 and thereby change the orientation of the displayed representations. For example, in the example shown in FIGS. 31 to 33, the sagittal normal line 248, best shown in the axial and coronal view panes 212, 216, is provided with at least one rotation handle 249 that is selectable by a user and usable to rotate the displayed normal lines about the marker device 242.

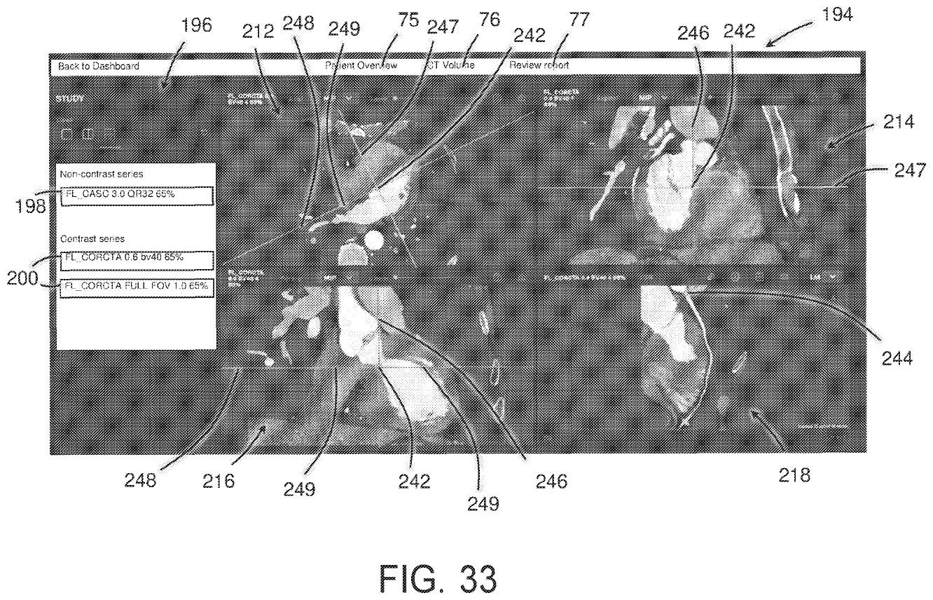

For example, as shown in FIG. 33, selection of a rotation handle 249 on the axial representation 234, in this example a rotation handle 249 disposed on the sagittal normal line 248, causes both the coronal normal line 247 and the sagittal normal line 248 to rotate about the marker device 242, and the representations shown in the sagittal and coronal view panes 214, 216 to change according to the respective planes that pass through the marker device normal to the coronal and sagittal normal lines 247, 248. In this example, similar rotation handles 249 are also disposed on the sagittal line 248 shown in the coronal view pane 216.

In this example, selection of a rotation handle 249 and rotation of the relevant normal lines may be effected by disposing a mouse pointer on a rotation handle 249, clicking on the mouse and simultaneously moving the mouse, although it will be understood that any suitable interface arrangement for achieving this is envisaged.

It will be understood that using the multi-view screen 195, a user is able to easily select a location of interest on a vessel, for example a stenotic location on the vessel, for example using the MPR view 240, and to display multiple desired views of the location of interest by causing display of selected locations along respective axial, coronal or sagittal normal lines, and/or changing the orientation of the axial, coronal or sagittal normal lines.

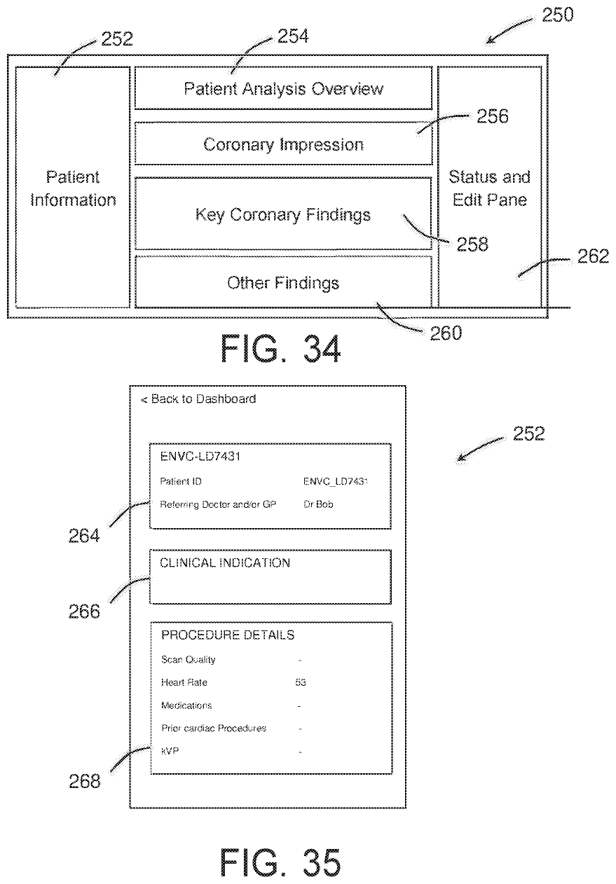

An example snapshot annotation screen 240 displayed in response to selection of a snapshot button 108, 132, 152 is shown in FIG. 30. Using the annotation screen 240, a user is able to add indicia such as annotation text 242 and an annotation arrow 244. Referring to the patient overview screen shown in FIG. 4, selection of the review report button 77 causes a report screen 250 shown in FIG. 34 to be displayed.

The report screen 250 includes a patient information pane 252, a patient analysis overview section 254, a coronary impression section 256, a key coronary findings section 258, an other findings section 260 and a status and edit pane 262.

An example representation of the patient information pane 252 is shown in FIG. 35. The patient information pane 252 includes a patient identification section, a clinical indication section 266 and a procedure details section 268.

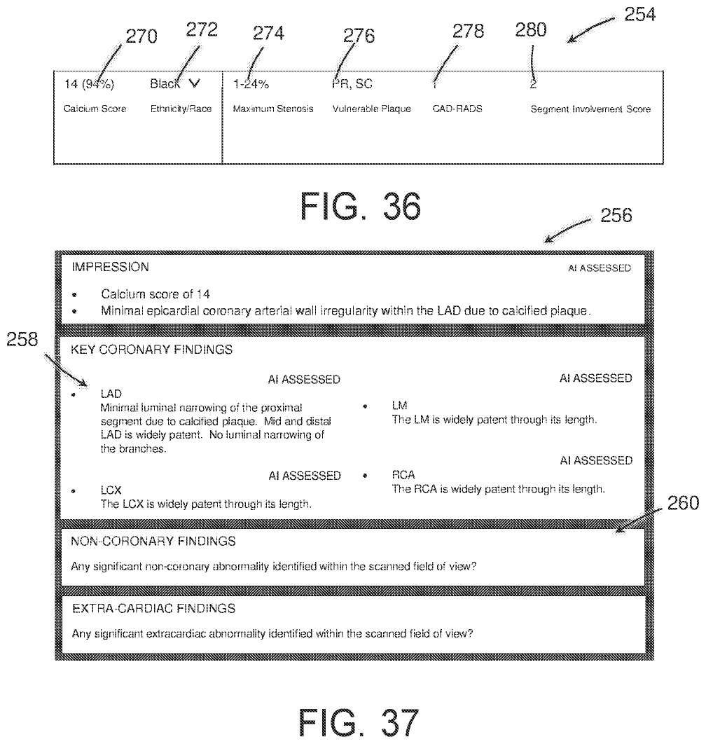

An example representation of the patient analysis overview section 254 is shown in FIG. 36. The patient analysis overview section 254 in this example includes a calcium score 270, ethnicity information 272, a maximum stenosis level 274, vulnerable plaque information 276, a CAD-RADS classification 278 and a segment involvement score 280.

An example representation of the coronary impression section 256, the key coronary findings section 258, and the other findings section 260 is shown in FIG. 37.

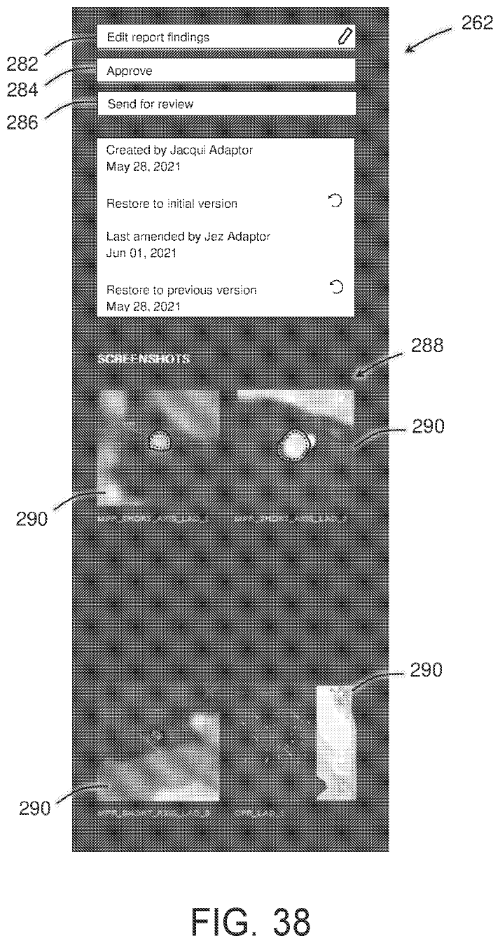

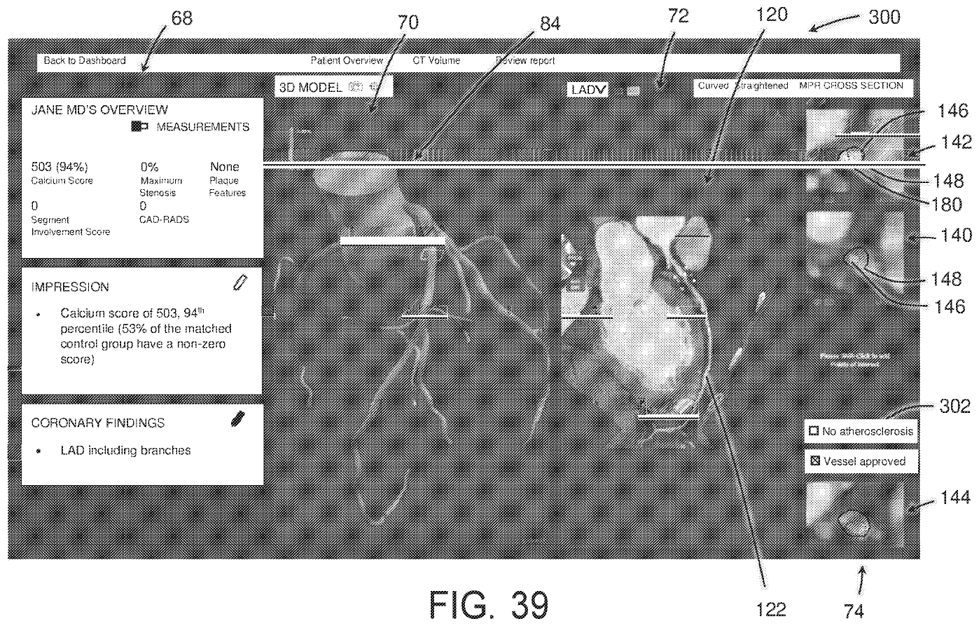

An example representation of the status and edit pane 262 is shown in FIG. 38. The status and edit pane 262 is used to facilitate editing of the report findings by selecting an edit report button 282, to change the status of the report using a report status box 286 and to approve the report using an approve box 284.

The status and edit pane 262 also includes screenshot tiles 290 associated with annotated screenshots.

The system is arranged such that editing of a report finding causes another report finding to also change if the findings are related and an amendment is required. For example, if a user modifies a stenosis level finding, and the new stenosis finding corresponds to a different CAD-RADs classification, the system also effects amendment of the CAD-RADs finding.

In an embodiment, operation of the analysis device 26 of the CAD analysis system 10 is also responsive to amendments made using the user interface 53.

For example, as described above, determination of coronary artery inner and outer walls and subsequent CAD analysis based on the walls may be implemented in response to user addition of a new centreline so that CAD analysis results of a coronary artery not initially identified by the analysis device 26 can be communicated to the user through the user interface 53.

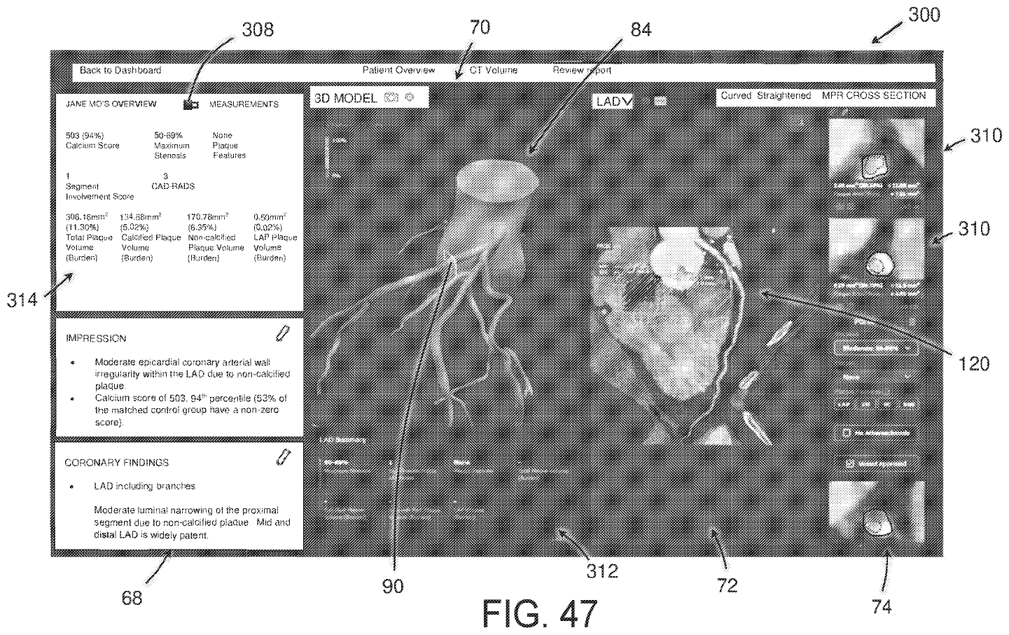

Example screens of a user interface of an alternate embodiment of a CAD analysis system are shown in FIGS. 39 to 47.

In this embodiment, the CAD analysis system is arranged to communicate disease assessment determinations, for example in relation to presence and severity of stenosis, calcium score calculation, and vulnerable plaque detection and characterisation, in response to user interaction. For example, in an embodiment, in response to an indication from a user that a stenosis lesion is present on a vessel, the system is arranged to display a stenosis lesion on the 3D model 84 and to display automatically generated stenosis information that includes a predicted stenosis level associated with the user identified stenosis lesion. The displayed stenosis information may be editable by the user.

In the present example, the CAD analysis system is arranged to automatically make disease assessment determinations, for example in relation to presence and severity of stenosis, calcium score calculation, and vulnerable plaque detection and characterisation, but at least some such determinations are only communicated to a user in response to user input.

FIG. 39 shows an alternative patent overview screen 300. Like and similar features are indicated with like reference numerals. As shown, prior to user interaction, the 3D model 84 does not include any information in relation to stenosis lesions that may be present on the coronary arteries 88, and no predictive information is provided for the slice representations 140, 142, 144.

The patient overview screen 300 includes an atherosclerosis selection box 302 usable to indicate that no atherosclerosis is considered to exist on a selected vessel 88 if the user considers that this is appropriate after the user has reviewed the vessel 88.

The user is able to select a location on the vessel 88 corresponding to a stenosis lesion, for example by right clicking at the relevant location on the MPR representation 120, if during a vessel review the user considers that a stenosis lesion exists on the vessel 88.

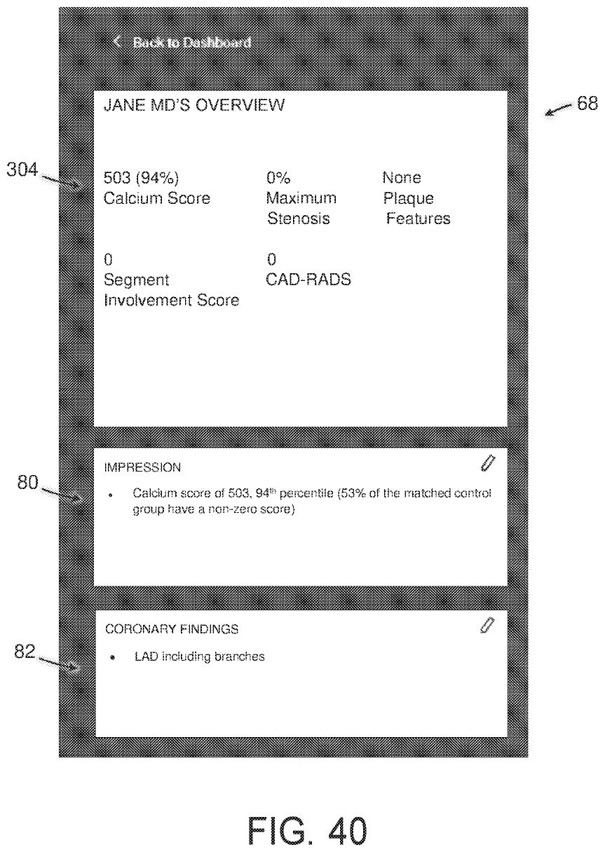

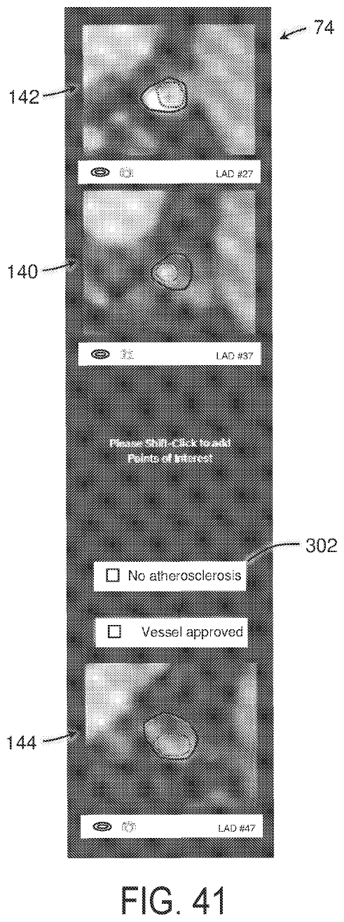

As shown in FIG. 40, prior to identification of any stenosis lesions by a user, a results summary section 304, that is similar to the results summary section 78 of the above embodiment, includes only total calcium score information. The remaining results summary information reflect that no stenosis lesions or vulnerable plaque (referred to in this embodiment as ‘plaque features’) have yet been identified by the user.

In this example, the results summary section 304 includes the following information fields:
a calcium (Agatston) score;
a maximum stenosis level that indicates the highest level of stenosis determined in the dataset;
an indication of the plaque features (if any) present in the dataset;
the relevant CAD-RADS classification; and
a segment involvement score that indicates how many stenosis lesions are present in the dataset.

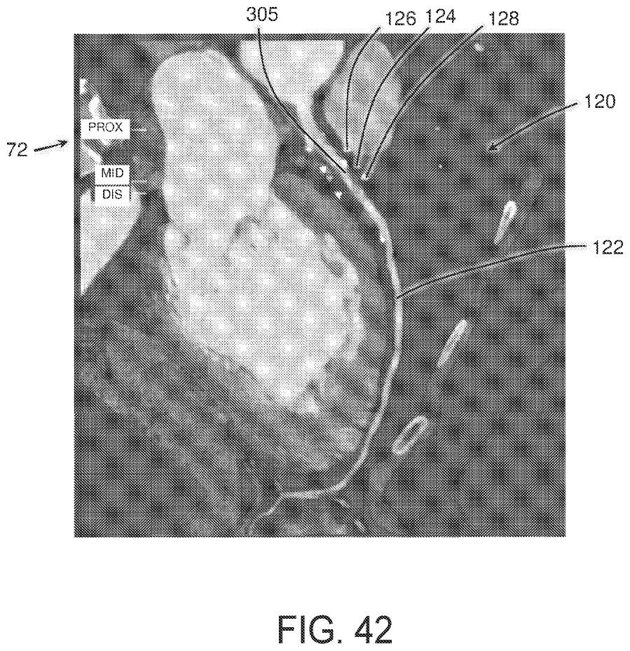

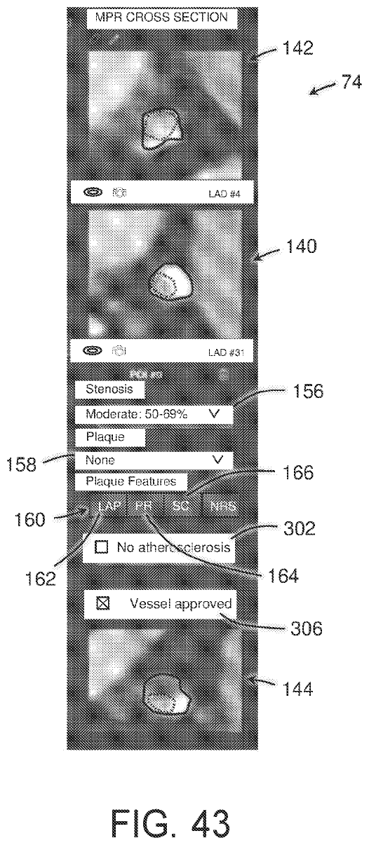

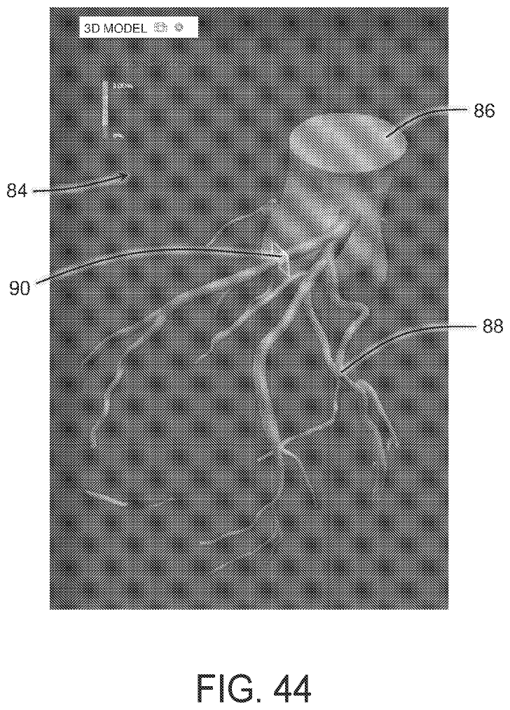

Enlarged views of the vessel slice pane 74 and the multiplanar reconstruction (MPR) pane 72 are shown in FIGS. 41 and 42. If the user selects a location 305 on the vessel 122 that is believed to correspond to a stenosis lesion, for example because the corresponding slice representation 140 is considered to show stenosis, the system automatically displays a stenosis lesion 90 on the 3D model 84 and associated stenosis lesion information adjacent the slice representation 140, as shown in FIGS. 43 and 44.

In this example, the predicted stenosis lesion characteristics are derived in the same way as in the above embodiment described in relation to FIGS. 3 to 38—using the analysis carried out on the patient CT dataset by the coronary artery analysis component 32.

However, instead of automatically displaying the vessel characteristics, including stenosis lesion characteristics, with the present embodiment the vessel characteristics are already determined but only displayed on the 3D model 84 and adjacent the slice representation 140 after the slice associated with the slice representation 140 has been selected by a user. However, it will be understood that other arrangements are possible. For example, in an alternate arrangement, the system is arranged to determine the relevant vessel characteristics, including stenosis lesion characteristics, only after the user has selected a vessel slice.

In the present example, the stenosis lesion characteristics include a stenosis level box 156 that indicates the maximum stenosis level of the stenosis lesion with which the selected slice is associated, a plaque type box 158 that indicates the type of plaque present in the stenosis lesion, and plaque feature labels 160 that indicate the type of vulnerable plaque present on the stenosis lesion. In the present example, the vulnerable plaque labels 160 include a low attenuation plaque label 162, a positive remodelling label 164, a spotty calcification label 166, and a napkin ring sign label 167.

As with the above embodiment, the colour of the stenosis lesion 90 and the stenosis level box 156 represent the severity of the stenosis lesion so that the user can quickly identify the stenosis level of the stenosis lesion 90 based colour.

In the present example, the added stenosis lesion has been automatically categorised by the system as ‘Moderate: 50-69%’ and no relevant plaque features are considered to exist.

The user may add further stenosis lesions 90 to the present coronary artery 88 until all relevant stenosis lesions are considered to have been identified, and the user then selects a vessel approved box 306 to indicate that the relevant vessel has been reviewed for the purpose of identifying stenosis lesions.

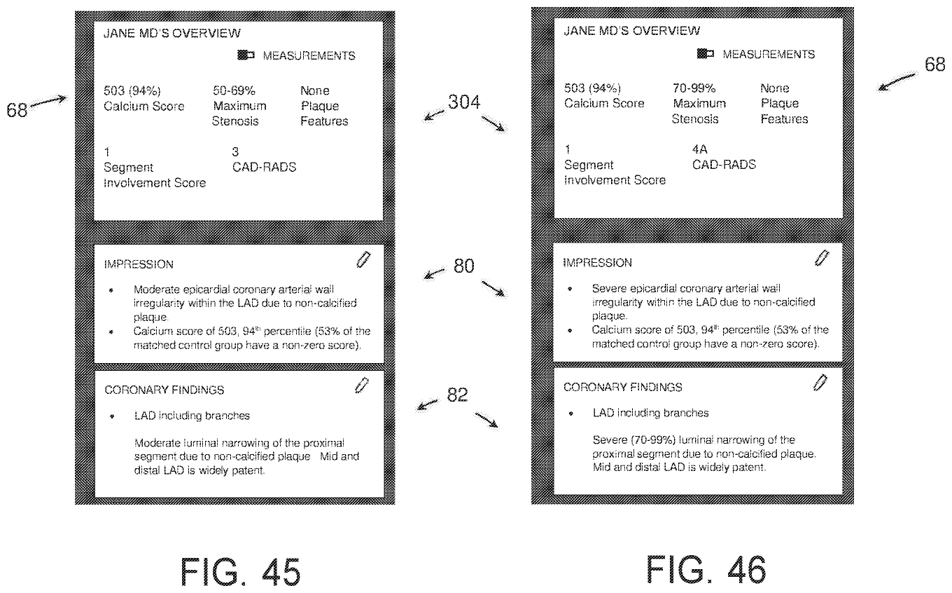

As shown in FIG. 45, after at least one stenosis lesion 90 has been added, the results summary screen 68 is automatically modified to include information associated with the stenosis lesion 90. Accordingly, in this example, the maximum stenosis level is now 50-69%, the plaque features are ‘none’, the relevant CAD-RADS classification is 3, and the segment involvement score is 1.

The stenosis lesion characteristics that are automatically determined and displayed after selection of a stenosis lesion by a user are editable by the user, and in response to user edits, the displayed stenosis lesion characteristics may change. In this example, in addition to the inner and outer walls and the vessel centrelines, the user is able to edit the stenosis level box 156, the plaque type box 158 and the plaque feature labels 160. For example, if the user edits the stenosis level box 156 to indicate that the stenosis lesion should be categorised as ‘Severe: 70-100%’ instead of ‘Moderate: 50-69%’, the colour of the stenosis lesion shown on the 3D model is caused to change to red, and the information on the results summary screen 68 also changes to reflect the user modified stenosis level, as shown in FIG. 46.

In this example, the patient overview screen 300 also includes a measurements toggle box that when selected causes automatically determined measurements to be displayed. In this example, the measurements include vessel and plaque slice area values 310, vessel summary information 312 and total plaque volume values 314.

In the claims which follow and in the preceding description, except where the context requires otherwise due to express language or necessary implication, the word “comprise” or variations such as “comprises” or “comprising” is used in an inclusive sense, i.e. to specify the presence of the stated features but not to preclude the presence or addition of further features in various embodiments.

Modifications and variations as would be apparent to a skilled addressee are deemed to be within the scope of the present invention.

1. A coronary artery disease (CAD) analysis system comprising:
a CAD analysis device arranged to analyse received patient CT scan data and produce CAD analysis data indicative of presence and characterisation of coronary artery disease in the patient CT scan data, the CAD analysis data indicative of at least one individual stenosis lesion on a coronary artery and a characterisation of the stenosis lesion;
the CAD analysis system arranged to identify a start location and an end location of the individual stenosis lesion on the coronary artery by identifying start and end slices of the individual stenosis lesion, and the CAD analysis system arranged to categorise all slices between the start and end slices as belonging to the individual stenosis lesion; and
the CAD analysis system comprising a user interface that displays a model of coronary arteries of a patient based on the patient CT scan data, the model configured to visually indicate an individual stenosis lesion and the individual stenosis lesion characterisation to a user so that the user is able to identify presence and characterisation of each individual stenosis lesion based on the visual indication;
wherein the characterisation of the individual stenosis lesion includes a stenosis level for the individual stenosis lesion.

2. A CAD analysis system as claimed in claim 1, wherein the characterisation also includes:
an indication of vulnerable plaque presence and vulnerable plaque type;
an indication of plaque presence and plaque type;
a lesion number; and/or
an artery slice number.

3. (canceled)

4. A CAD analysis system as claimed in claim 1, wherein the stenosis level is visually communicated by displaying a portion of a coronary artery associated with the individual stenosis lesion in a defined colour of a plurality of colours of a stenosis level colour key, the defined colour corresponding to the stenosis level of the lesion.

5. A CAD analysis system as claimed in claim 1, wherein:
the visual indication of the individual stenosis lesion and the individual stenosis lesion characterisation are displayed in response to user input; or
the visual indication of the individual stenosis lesion and the individual stenosis lesion characterisation are displayed in response to user selection of a location on a coronary artery considered to correspond to a stenosis lesion.

6-11. (canceled)

12. A CAD analysis system as claimed in claim 1, wherein the user interface includes a multiplanar reconstruction (MPR) representation of at least a selected coronary artery.

13. A CAD analysis system as claimed in claim 12, wherein the model of coronary arteries includes a first vessel slice identifier arranged to indicate a selected slice of a coronary artery, the MPR representation includes a second vessel slice identifier arranged to indicate a selected slice of the selected coronary artery, and
the first and second vessel slice identifiers are synchronised such that selection of a vessel slice using one of the first and second vessel slice identifiers causes a corresponding vessel slice to be selected using the other of the first and second vessel slice identifiers.

14-16. (canceled)

17. A CAD analysis system as claimed in claim 1, wherein the user interface further includes an axial slice representation of a selected coronary artery at a selected coronary artery slice and the axial slice representation includes inner and outer vessel wall annotations.

18. (canceled)

19. A CAD analysis system as claimed in claim 17, wherein, for the axial slice representation, the user interface further includes stenosis lesion specific information for the stenosis lesion with which the selected slice is associated, the lesion specific information associated with the selected slice including:
a stenosis level of the stenosis lesion with which the selected slice is associated;
plaque type information for plaque present on the stenosis lesion with which the selected slice is associated; and/or
vulnerable plaque type information for vulnerable plaque present on the stenosis lesion with which the selected slice is associated.

20. (canceled)

21. A CAD analysis system as claimed in claim 19, wherein the user interface includes a multiplanar reconstruction (MPR) representation of at least a selected coronary artery, and wherein the stenosis level of the stenosis lesion with which the selected slice is associated is communicated by displaying a portion of the MPR representation corresponding to the stenosis lesion using a colour used to display the stenosis lesion on the model of the coronary arteries, and/or by displaying text indicative of the stenosis level using the colour used to display the stenosis lesion on the model of the coronary arteries, and/or by displaying at least a portion of the selected slice using the colour used to display the stenosis lesion on the model of the coronary arteries.

22-25. (canceled)

26. A CAD analysis system as claimed in claim 1, wherein the user interface is arranged to display a coronary artery centreline, the path of the centreline, being editable by a user.

27. (canceled)

28. A CAD analysis system as claimed in claim 26, wherein the user interface is arranged to enable a user to add a new centreline associated with a coronary artery.

29. A CAD analysis system as claimed in claim 1, wherein the user interface is arranged to display representations of calcified volumes on the model of coronary arteries in response to user input, and to display information indicative of locations of vulnerable plaque on the model of coronary arteries in response to user input.

30-34. (canceled)

35. A CAD analysis system as claimed in claim 1, wherein the user interface is arranged to simultaneously display multiple CT volume representations of a CT volume associated with the patient CT scan data, each CT volume representation taken along a plane extending through the CT volume at a different orientation, and the multiple displayed CT volume representations having a common CT volume location.

36-37. (canceled)

38. A CAD analysis system as claimed in claim 35, wherein the interface is arranged to display plane indicia on a CT volume representation, the plane indicia indicative of a plane associated with another CT volume representation, and to enable a user to interact with the plane indicia to modify the plane associated with the other CT volume representation and thereby the displayed other CT volume representation.

39-42. (canceled)

43. A CAD analysis system as claimed in claim 35, wherein the user interface is arranged to display a vessel MPR representation of a selected vessel with the multiple CT volume representations of the CT volume, wherein the common CT volume location is a selected location on the vessel MPR.

44. A CAD analysis system as claimed in claim 43, wherein the user interface is arranged to enable a user to move the selected location on the vessel MPR, and to change the multiple displayed CT volume representations of the CT volume in synchronisation with the selected location on the vessel MPR so that the common CT location changes in accordance with the moved location on the vessel MPR.

45. A CAD analysis system as claimed in claim 1, wherein the CAD analysis system is arranged to enable a user to modify and/or add to displayed CAD analysis information, and in response the analysis device is arranged to reanalyse the patient CT scan data in consideration of the modification and/or addition.

46-49. (canceled)

50. A coronary artery disease (CAD) analysis system, the CAD analysis system comprising:
a CAD analysis device arranged to receive CAD data indicative of presence of calcified plaque on coronary arteries in patient CT scan data; and
a user interface arranged to:
display a scroll bar having a user controllable position indicator and coronary artery calcium indicia disposed adjacent the scroll bar, the location of the position indicator relative to the scroll bar indicating a respective position along an axis of a CT volume associated with the patient CT scan data, and each coronary artery calcium indicium indicative of a total amount of coronary artery calcification at an axial location of the CT scan data corresponding to the relative position of the position indicator on the scroll bar; and
display information indicative of coronary artery calcification at an axial location of the CT volume and associated with a coronary artery calcium indicium when the position indicator is disposed adjacent the coronary artery calcium indicium.

51. A CAD analysis system as claimed in claim 50, wherein each calcium indicium includes a graphical indicator, wherein a dimension of the graphical indicator is indicative of a size of the associated calcified volume.

52. (canceled)

53. A CAD analysis system as claimed in claim 50, wherein a colour of the calcium indicium is indicative of a coronary artery on which the calcified volume is located.

54. (canceled)

55. A CAD analysis system as claimed in claim 50, wherein a vessel label is displayed adjacent a displayed calcified volume, and the coronary artery label associated with a displayed calcified volume is editable by a user to change the associated coronary artery to a different coronary artery.

56-57. (canceled)

58. A CAD analysis system as claimed in claim 50, wherein non-coronary artery calcium is displayed in a different colour to the calcified volumes displayed on the coronary arteries.
